(19) United States
(12) Patent Application Publication
(10) Pub. No.: US 2001/0046486 A1
(43) Pub. Date: Nov. 29, 2001

(54) Stimulation of thymus for vaccination development

(76) Inventor: Richard L. Boyd, Melbourne (AU)

Correspondence Address: IRELL & MANELLA LLP, 1800 AVENUE OF THE STARS, SUITE 900, LOS ANGELES, CA, 90067, US

(21) Appl. No.: 09/755,965

(22) Filed: Jan. 5, 2001

Related U.S. Application Data
(63) Continuation-in-part of application No. PCT/AU00/00329, filed on Apr. 17, 2000.

(51) Int. Cl. A61K 45/00

(52) U.S. Cl. 424/93.7

(57) ABSTRACT

The present invention provides methods for enhancing the response of a patient's immune system to vaccination. This is accomplished by reactivating the thymus. Optionally, hematopoietic stem cells, autologous, syngeneic, allogeneic or xenogeneic, are delivered to increase the speed of regeneration of the patient's immune system. In a preferred embodiment the hematopoietic stem cells are CD34⁺. The patient's thymus is reactivated by disruption of sex steroid signaling to the thymus. In a preferred embodiment, this disruption is created by administration of LHRH agonists, LHRH antagonists, anti-LHRH receptor antibodies, anti-LHRH vaccines or combinations thereof.

This application is a continuation-in-part of Australian Patent Application PR0745, filed Oct. 13, 2000, as well as of U.S. Ser. No. 60/240,588, filed October 13, 2000, and U.S. Ser. No. 60/240,586, filed Oct. 13, 2000, both of which are continuation-in-part applications of PCT/AU00/00329, filed Apr. 17, 2000, which is an international filing of Australian patent application PP9778, filed Apr. 15, 1999, each of which is incorporated herein by reference.

### FIELD OF THE INVENTION

The present invention is in the field of response to vaccines in animals. More particularly, the present invention is in the field of improving vaccine response, through stimulation of the thymus.

### BACKGROUND

### THE IMMUNE SYSTEM

The major function of the immune system is to distinguish “foreign” antigens from “self” and respond accordingly to protect the body against infection. This definition may also be stated as distinguishing “bad” molecules from “good.” In normal immune responses, the sequence of events involves dedicated antigen presenting cells (APC) capturing foreign antigen and processing it into small peptide fragments which are then presented in clefts of major histocompatibility complex (MHC) molecules on the APC surface. The MHC molecules can either be of class I expressed on all nucleated cells (recognized by cytotoxic T lymphocytes (Tc)) or of class II expressed primarily by cells of the immune system (recognized by helper T lymphocytes (Th)).

Resting APC are programmed for antigen trapping and processing into peptide fragments, which are then expressed on the surface MHC molecules. These more activated APC then become better able to stimulate T cells rather than trap and process additional antigens. Th cells recognize the MHC II/peptide complexes on APC and respond.

There are two types of Th cells distinguished by the different types of soluble regulatory factors they produce. Th1 cells primarily produce IL2 and gamma interferon (IFNγ). The latter, if present at the time of initial contact of naïve T cells with antigen, promotes the preferential activation of cell-mediated immunity (primarily Tc). Th2 cells express a different profile of cytokines, especially IL4, 5 and 10, which induce humoral immunity via antibody producing B cells that are specific for the particular antigen. In some cases this can lead to inappropriate allergic responses through IgE production.

The importance of Th cells in virtually all immune responses is best illustrated in HIV/AIDS where their absence through destruction by the virus causes severe immune deficiency, eventually leading to death. Given their central importance as immunoregulatory cells, the balance between Th1 and Th2 cells can have profound impact on the nature of the immune response. Such imbalances can occur through developmental abnormalities or inappropriate activation at the onset of immune responses. This can lead to a variety of diseases such as allergies, cancer and autoimmunity.

### THE THYMUS

The thymus is arguably the major organ in the immune system because it is the primary site of production of T lymphocytes. Its role is to attract appropriate bone marrow-derived precursor cells from the blood, and induce their commitment to the T cell lineage including the gene rearrangements necessary for the production of the T cell receptor for antigen (TCR). Associated with this is a remarkable degree of cell division to expand the number of T cells and thereby increase the likelihood that every foreign antigen will be recognized and eliminated. This enormous potential diversity means that for any single antigen the body might encounter, multiple lymphocytes will be able to recognize it with varying degrees of binding strength (affinity) and respond to varying degrees. A strange feature of T cell recognition of antigen, however, is that unlike B cells, the TCR only recognizes peptide fragments physically associated with MHC molecules; normally this is self MHC and this ability is selected for in the thymus. This process is called positive selection and is an exclusive feature of cortical epithelial cells. If the TCR fails to bind to the self MHC/peptide complexes, the T cell dies by “neglect.” It needs some degree of signaling through the TCR for its continued maturation.

Following selection in the cortex, the developing thymocytes acquire functional maturation and migratory capacity and exit into the blood stream as naïve (not yet having contacted antigen) T cells. They circulate between the lymph and blood in search of antigen. If, after 3-4 weeks, they haven't been stimulated, they become susceptible to deletion from the peripheral T cell pool by other recent thymic emigrants. This system of thymic export and peripheral T cell replacement provides a continual replenishment of the quality of T cells, with homeostasis maintaining the appropriate levels.

While the thymus is fundamental for a functional immune system, releasing about 1% of its T cell content into the bloodstream per day, one of the apparent anomalies of mammals is that this organ undergoes severe atrophy as a result of sex steroid production. This can begin even in young children but is profound from the time of puberty. For normal healthy individuals this loss of production and release of new T cells does not always have clinical consequences. In fact, although the aged thymus is atrophic and consists of less than 1% of its young counterpart (see below), it still continues to release a very low level of new T cells into the blood stream. While these are insufficient to maintain the optimal levels of peripheral T cell subsets, the thymus is not completely dormant, raising the possibility that it could be the target of therapy.

With progressive aging, the decline in thymic export means that the status of peripheral T cells undergoes progressive change, both quantitatively and qualitatively. In addition to a gradual decrease in absolute T cell numbers in the blood with age as they die off through lack of stimulation, with each antigen contact the relevant antigen-specific naïve T cells (those that have not yet encountered antigen) are stimulated and proliferate. A subset will progress to be effector cells to remove the pathogen, but these eventually die through antigen-induced cell death.

Another subset will convert to memory cells and provide long term protection against future contacts with that pathogen. Thus, there is a decrease in the levels of naïve T cells and, as a result, a reduced ability to respond to antigen. Aging also results in a selective decline in Th cells (characterized by expression of CD4) relative to Tc cells (expressing CD8), and imbalances in the ratios of Th1 to Th2 cells. This does not occur in the normal young because, as mentioned above, there is a continual supply of new T cells being exported from the thymus, which in turn provides a continual replenishment of the naïve T cell pool in the periphery.

### THYMUS ATROPHY

The thymus is influenced to a great extent by its bidirectional communication with the neuroendocrine system (Kendall, 1988). Of particular importance is the interplay between the pituitary, adrenals and gonads on thymic function including both trophic (thyroid stimulating hormone or TSH and growth hormone or GH) and atrophic effects (leutinizing hormone or LH, follicle stimulating hormone or FSH, and adrenocorticotropic hormone or ACTH) (Kendall, 1988; Homo-Delarche, 1991). Indeed one of the characteristic features of thymic physiology is the progressive decline in structure and function which is commensurate with the increase in circulating sex steroid production around puberty (Hirokawa and Makinodan, 1975; Tosi et al., 1982 and Hirokawa, et al., 1994). The precise target of the hormones and the mechanism by which they induce thymus atrophy is yet to be determined. Since the thymus is the primary site for the production and maintenance of the peripheral T cell pool, this atrophy has been widely postulated as the primary cause of an increased incidence of immune-based disorders in the elderly. In particular, deficiencies of the immune system illustrated by a decrease in T-cell dependent immune functions such as cytolytic T-cell activity and mitogenic responses, are reflected by an increased incidence of immunodeficiency, autoimmunity and tumor load in later life (Hirokawa, 1998).

The impact of thymus atrophy is reflected in the periphery, with reduced thymic input to the T cell pool resulting in a less diverse T cell receptor (TCR) repertoire. Altered cytokine profile (Hobbs et al., 1993; Kurashima et al., 1995), changes in CD4⁺ and CD8⁺ subsets and a bias towards memory as opposed to naïve T cells (Mackall et al., 1995) are also observed. Furthermore, the efficiency of thymopoiesis is impaired with age such that the ability of the immune system to regenerate normal T-cell numbers after T-cell depletion, is eventually lost (Mackall et al., 1995). However, recent work by Douek et al. (1998), has shown presumably thymic output to occur even in old age in humans. Excisional DNA products of TCR gene-rearrangement were used to demonstrate circulating, de novo produced naïve T cells after HIV infection in older patients. The rate of this output and subsequent peripheral T cell pool regeneration needs to be further addressed since patients who have undergone chemotherapy show a greatly reduced rate of regeneration of the T cell pool, particularly CD4⁺ T cells, in post-pubertal patients compared to those who were pre-pubertal (Mackall et al, 1995). This is further exemplified in recent work by Timm and Thoman (1999), who have shown that although CD4⁺ T cells are regenerated in old mice post bone marrow transplant (BMT), they appear to show a bias towards memory cells due to the aged peripheral microenvironment, coupled to poor thymic production of naïve T cells.

The thymus essentially consists of developing thymocytes interspersed within the diverse stromal cells (predominantly epithelial cell subsets) which constitute the microenvironment and provide the growth factors and cellular interactions necessary for the optimal development of the T cells. The symbiotic developmental relationship between thymocytes and the epithelial subsets that controls their differentiation and maturation (Boyd et al., 1993), means sex-steroid inhibition could occur at the level of either cell type which would then influence the status of the other. It is less likely that there is an inherent defect within the thymocytes themselves since previous studies, utilizing radiation chimeras, have shown that bone marrow (BM) stem cells are not affected by age (Hirokawa, 1998; Mackall and Gress, 1997) and have a similar degree of thymus repopulation potential as young BM cells. Furthermore, thymocytes in older aged animals retain their ability to differentiate to at least some degree (Mackall and Gress, 1997; George and Ritter, 1996; Hirokawa et al., 1994). However, recent work by Aspinall (1997), has shown a defect within the precursor CD3⁻CD4⁻CD8⁻ triple negative (TN) population occurring at the stage of TCRγ chain gene-rearrangement.

Aging is not the only condition that results in T cell loss; this also occurs very severely, for example, in HIV/AIDS and following chemotherapy or radiotherapy. Again, in the young with an active thymus, recovery of the immune system (through recovery of T cell mediated immunity) occurs relatively quickly (2-3 months) compared to post-puberty when it can take 1-2 years because of the atrophic thymus.

### VACCINES

Vaccines can be divided into two classes: those that provide active vaccination and those that provide passive vaccination. Passive vaccination involves the administration to a patient of antibodies from a heterologous source to react against foreign antigens in the patient or that the patient will encounter. Such vaccination is usually very short lived, as the native immune system of the patient is not involved. The present invention is in the field of active vaccinations, where an antigen is administered to a patient whose immune system then responds to the antigen by forming antibodies specific to the antigen.

There are several parameters that can influence the nature and extent of immune responses: the level and type of antigen, the site of vaccination, the availability of appropriate APC, the general health of the individual, and the status of the T and B cell pools. Of these, T cells are the most vulnerable because of the marked sex steroid-induced shutdown in thymic export that becomes profound from the onset of puberty. Any vaccination program should therefore only be logically undertaken when the level of potential responder T cells is optimal with respect to both the existence of naïve T cells representing a broad repertoire of specificity, and the presence of normal ratios of Th1 to Th2 cells and Th to Tc cells. The level and type of cytokines should also be manipulated to be appropriate for the desired response.

The ability to reactivate the atrophic thymus through inhibition of sex steroid production, for example at the level of leutinizing hormone releasing hormone (LHRH) signaling to the pituitary, provides a potent means of generating a new cohort of naïve T cells with a diverse repertoire of TCR types. This process effectively reverts the thymus to its pre-pubertal state and does so by using the normal regulatory molecules and pathways which lead to optimal thymopoiesis.

### SUMMARY OF THE INVENTION

The present invention concerns methods for improving an animal's immune response to a vaccine. This is accomplished by quantitatively and qualitatively restoring the peripheral T cell pool, particularly at the level of naïve T cells. These naïve T cells are then able to respond to a greater degree to presented foreign antigen.

The methods of this invention rely on blocking sex steroid signaling to the thymus. In a preferred embodiment, chemical castration is used. In another embodiment surgical castration is used. Castration reverses the state of the thymus to its pre-pubertal state, thereby reactivating it.

In a particular embodiment sex steroid signaling to the thymus is blocked by the administration of agonists or antagonists of LHRH, anti-estrogen antibodies, anti-androgen antibodies, or passive (antibody) or active (antigen) anti-LHRH vaccinations.

### FIGURES

FIG. 1: Changes in thymocyte number pre- and post-castration. Thymus atrophy results in a significant decrease in thymocyte numbers with age. By 2 weeks post-castration, cell numbers have increased to young adult levels. By 3 weeks post-castration, numbers have significantly increased from the young adult and they are stabilized by 4 weeks post-castration. ***=Significantly different from young adult (2 month) thymus, p<0.001

FIG. 2: (A) Spleen numbers remain constant with age and post-castration. The B:T cell ratio in the periphery also remains constant (B), however, the CD4:CD8 ratio decreases significantly (p<0.001) with age and is restored to normal young levels by 4 weeks post-castration.

FIG. 3: Fluorescence Activated Cell Sorter (FACS) profiles of CD4 vs. CD8 thymocyte populations with age and post-castration. Percentages for each quadrant are given above each plot. Subpopulations of thymocytes remain constant with age and there is a synchronous expansion of thymocytes following castration.

FIG. 4A: Proliferation of thymocytes as detected by incorporation of a pulse of BrdU. Proportion of proliferating thymocytes remains constant with age and following castration.

FIG. 4B: Effects of age and castration on proliferation of thymocyte subsets. (A) Proportion of each subset that constitutes the total proliferating population—The proportion of CD8+T cells within the proliferating population is significantly increased. (B) Percentage of each subpopulation that is proliferating—The TN and CD8 Subsets have significantly less proliferation at 2 years than at 2 months. At 2 weeks post-castration, the TN population has returned to normal young levels of proliferation while the CD8 population shows a significant increase in proliferation. The level is equivalent to the normal young by 4 weeks post-castration. (C) Overall TN proliferation remains constant with age and post-castration, however, the significant decrease in proliferation of the TN1 subpopulation with age, is not returned to normal levels by 4 weeks post-castration (D). ***=Highly significant, p<0.001, **=significant, p<0.01

FIG. 5: Mice were injected intrathymically with FITC. The number of FITC+ cells in the periphery were calculated 24 hours later. Although the proportion of recent thymic migrants (RTE) remained consistently about 1% of thymus cell number age but was significantly reduced at 2 weeks post-castration, there was a significant (p<0.01) decrease in the RTE cell numbers with age. Following castration, these values were increasing although still significantly lower than young mice at 2 weeks post-castration. With age, a significant increase in the ratio of CD4+ to CD8+ RTE was seen and this was normalized by 1 week post-castration.

FIG. 6: Changes in thymus, spleen and lymph node cell numbers following treatment with cyclophosphamide, a chemotherapy agent. Note the rapid expansion of the thymus in castrated animals when compared to the non-castrate (cyclophosphamide alone) group at 1 and 2 weeks post-treatment. In addition, spleen and lymph node numbers of the castrate group were well increased compared to the cyclophosphamide alone group. By 4 weeks, cell numbers are normalized. (n=3-4 per treatment group and time point).

FIG. 7: Changes in thymus, spleen and lymph node cell numbers following irradiation (625 Rads) one week after surgical castration. Note the rapid expansion of the thymus in castrated animals when compared to the non-castrate (irradiation alone) group at 1 and 2 weeks post-treatment. (n=3-4 per treatment group and time point).

FIG. 8: Changes in thymus, spleen and lymph node cell numbers following irradiation and castration on the same day. Note the rapid expansion of the thymus in castrated animals when compared to the non-castrate group at 2 weeks post-treatment. However, the difference observed is not as obvious as when mice were castrated 1 week prior to treatment (FIG. 7). (n=3-4 per treatment group and time point).

FIG. 9: Changes in thymus, spleen and lymph node cell numbers following treatment with cyclophosphamide, a chemotherapy agent, and surgical or chemical castration performed on the same day. Note the rapid expansion of the thymus in castrated animals when compared to the non-castrate (cyclophosphamide alone) group at 1 and 2 weeks post-treatment. In addition, spleen and lymph node numbers of the castrate group were well increased compared to the cyclophosphamide alone group. (n=3-4 per treatment group and time point). Chemical castration is comparable to surgical castration in regeneration of the immune system post-cyclophosphamide treatment.

FIG. 10: Lymph node cellularity following foot-pad immunization with Herpes Simplex Virus-1(HSV-1). Note the increased cellularity in the aged post-castration as compared to the aged non-castrated group. Bottom graph illustrates the overall activated cell number as gated on CD25 vs. CD8 cells by FACS.

FIG. 11: Representative examples of flow cytometry dot plots illustrating activated cell proportions in lymph nodes following Herpes Simplex Virus-1(HSV-1) infection. Activated cells are CD25+CD8+ “Non-immune” are pooled popliteal lymph node cells from control uninfected young (2 months); “old immune” are pooled popliteal lymph node cells from uninfected old (˜18 months old) mice. “Young immune” is a representative example a popliteal lymph node from a young mice infected 5 days earlier with HSV-1 in the lower hind leg.

FIG. 12A: Vβ10 expression on CTL in activated LN following HSV-1 inoculation. Note the diminution of a clonal response in aged mice and the reinstatement of the expected response post-castration.

FIG. 12B: Popliteal lymph nodes were removed from mice immunized with HSV-1 and cultured for 3 days. CTL assays were performed with non-immunized mice as control for background levels of lysis (as determined by ⁵¹Cr-release). Results are expressed as mean of 8 mice, in triplicate ±1SD. Aged mice showed a significant (p 0.01, *) reduction in CTL activity at an E:T ratio of both 10:1 and 3:1 indicating a reduction in the percentage of specific CTL present within the lymph nodes. Castration of aged mice restored the CTL response to young adult levels.

FIG. 13: Changes in thymus, spleen, lymph node and bone marrow cell numbers following bone marrow transplantation of Ly5 congenic mice. Note the rapid expansion of the thymus in castrated animals when compared to the non-castrate group at all time points post-treatment. In addition, spleen and lymph node numbers of the castrate group were well increased compared to the cyclophosphamide alone group. (n=3-4 per treatment group and time point). Castrated mice had significantly increased congenic (Ly5.2) cells compared to non-castrated animals (data not shown).

FIG. 14: Changes in thymus cell number in castrated and noncastrated mice after fetal liver reconstitution. (n=3-4 for each test group.) (A) At two weeks, thymus cell number of castrated mice was at normal levels and significantly higher than that of noncastrated mice (*p≦0.05). Hypertrophy was observed in thymuses of castrated mice after four weeks. Noncastrated cell numbers remain below control levels. (B) CD45.2⁺ cells-CD45.2⁺ is a marker showing donor derivation. Two weeks after reconstitution donor-derived cells were present in both castrated and noncastrated mice. Four weeks after treatment approximately 85% of cells in the castrated thymus were donor-derived. There were no donor-derived cells in the noncastrated thymus.

FIG. 15: FACS profiles of CD4 versus CD8 donor derived thymocyte populations after lethal irradiation and fetal liver reconstitution, followed by surgical castration. Percentages for each quadrant are given to the right of each plot. The age matched control profile is of an eight month old Ly5.1 congenic mouse thymus. Those of castrated and noncastrated mice are gated on CD45.2⁺ cells, showing only donor derived cells. Two weeks after reconstitution subpopulations of thymocytes do not differ between castrated and noncastrated mice.

FIG. 16: Myeloid and lymphoid dendritic cell (DC) number after lethal irradiation, fetal liver reconstitution and castration. (n=3-4 mice for each test group.) Control (white) bars on the following graphs are based on the normal number of dendritic cells found in untreated age matched mice. (A) Donor-derived myeloid dendritic cells—Two weeks after reconstitution DC were present at normal levels in noncastrated mice. There were significantly more DC in castrated mice at the same time point. (*p≦0.05). At four weeks DC number remained above control levels in castrated mice. (B) Donor-derived lymphoid dendritic cells—Two weeks after reconstitution DC numbers in castrated mice were double those of noncastrated mice. Four weeks after treatment DC numbers remained above control levels.

FIG. 17: Changes in total and CD45.2⁺ bone marrow cell numbers in castrated and noncastrated mice after fetal liver reconstitution. n=3-4 mice for each test group. (A) Total cell number—Two weeks after reconstitution bone marrow cell numbers had normalized and there was no significant difference in cell number between castrated and noncastrated mice. Four weeks after reconstitution there was a significant difference in cell number between castrated and noncastrated mice (*p≦0.05). (B) CD45.2⁺ cell number. There was no significant difference between castrated and noncastrated mice with respect to CD45.2⁺ cell number in the bone marrow two weeks after reconstitution. CD45.2⁻ cell number remained high in castrated mice at four weeks. There were no donor-derived cells in the noncastrated mice at the same time point.

FIG. 18: Changes in T cells and myeloid and lymphoid derived dendritic cells (DC) in bone marrow of castrated and noncastrated mice after fetal liver reconstitution. (n=3-4 mice for each test group.) Controls (white) bars on the following graphs are based on the normal number of T cells and dendritic cells found in untreated age matched mice. (A) T cell number—Numbers were reduced two and four weeks after reconstitution in both castrated and noncastrated mice. (B) Donor derived myeloid dendritic cells—Two weeks after reconstitution DC cell numbers were normal in both castrated and noncastrated mice. At this time point there was no significant difference between numbers in castrated and noncastrated mice. (C) Donor-derived lymphoid dendritic cells—Numbers were at normal levels two and four weeks after reconstitution. At two weeks there was no significant difference between numbers in castrated and noncastrated mice.

FIG. 19: Change in total and donor (CD45.2⁺) spleen cell numbers in castrated and noncastrated mice after fetal liver reconstitution. (n=3-4 mice for each test group.) (A) Total cell number—Two weeks after reconstitution cell numbers were decreased and there was no significant difference in cell number between castrated and noncastrated mice. Four weeks after reconstitution cell numbers were approaching normal levels in castrated mice. (B) CD45.2⁺ cell number—There was no significant difference between castrated and noncastrated mice with respect to CD45.2⁺ cell number in the spleen, two weeks after reconstitution. CD45.2⁺ cell number remained high in castrated mice at four weeks. There were no donor-derived cells in the noncastrated mice at the same time point.

FIG. 20: Splenic T cells and myeloid and lymphoid derived dendritic cells (DC) after fetal liver reconstitution. (n=3-4 mice for each test group.) Control (white) bars on the following graphs are based on the normal number of T cells and dendritic cells found in untreated age matched mice. (A) T cell number—Numbers were reduced two and four weeks after reconstitution in both castrated and noncastrated mice. (B) Donor derived (CD45.2⁺) myeloid dendritic cells—two and four weeks after reconstitution DC numbers were normal in both castrated and noncastrated mice. At two weeks there was no significant difference between numbers in castrated and noncastrated mice. (C) Donor-derived (CD45.2⁺) lymphoid dendritic cells—numbers were at normal levels two and four weeks after reconstitution. At two weeks there was no significant difference between numbers in castrated and noncastrated mice.

FIG. 21: Changes in total and donor (CD45.2⁺) lymph node cell numbers in castrated and noncastrated mice after fetal liver reconstitution. (n=3-4 for each test group.) (A) Total cell numbers—Two weeks after reconstitution cell numbers were at normal levels and there was no significant difference between castrated and noncastrated mice. Four weeks after reconstitution cell numbers in castrated mice were at normal levels. (B) CD45.2⁺ cell number—There was no significant difference between castrated and noncastrated mice with respect to donor CD45.2⁺ cell number in the lymph node two weeks after reconstitution. CD45.2 cell number remained high in castrated mice at four weeks. There were no donor-derived cells in the noncastrated mice at the same point.

FIG. 22: Changes in T cells and myeloid and lymphoid derived dendritic cells (DC) in the mesenteric lymph nodes of castrated and non-castrated mice after fetal liver reconstitution. (n=3-4 mice for each test group.) Control (white) bars are the number of T cells and dendritic cells found in untreated age matched mice. (A) T cell numbers were reduced two and four weeks after reconstitution in both castrated and noncastrated mice. (B) Donor derived myeloid dendritic cells were normal in both castrated and noncastrated mice. At four weeks they were decreased. At two weeks there was no significant difference between numbers in castrated and noncastrated mice. (C) Donor-derived lymphoid dendritic cells—Numbers were at normal levels two and four weeks after reconstitution. At two weeks there was no significant difference between numbers in castrated and noncastrated mice.

FIG. 23: The phenotypic composition of peripheral blood lymphocytes was analyzed in patients (all>60 years) undergoing LHRH agonist treatment for prostate cancer. Patient samples were analyzed before treatment and 4 months after beginning LHRH agonist treatment. Total lymphocyte cell numbers per ml of blood were at the lower end of control values before treatment in all patients. Following treatment, 6/9 patients showed substantial increases in total lymphocyte counts (in some cases a doubling of total cells was observed). Correlating with this was an increase in total T cell numbers in 6/9 patients. Within the CD4⁺ subset, this increase was even more pronounced with 8/9 patients demonstrating increased levels of CD4 T cells. A less distinctive trend was seen within the CD8⁺ subset with 4/9 patients showing increased levels; albeit generally to a smaller extent than CD4⁺ T cells.

FIG. 24: Analysis of patient blood before and after LHRH-agonist treatment demonstrated no substantial changes in the overall proportion of T cells, CD4 or CD8 T cells, and a variable change in the CD4:CD8 ratio following treatment. This indicates the minimal effect of treatment on the homeostatic maintenance of T cell subsets despite the substantial increase in overall T cell numbers following treatment. All values were comparative to control values.

FIG. 25: Analysis of the proportions of B cells and myeloid cells (NK, NKT and macrophages) within the peripheral blood of patients undergoing LHRH agonist treatment demonstrated a varying degree of change within subsets. While NK, NKT and macrophage proportions remained relatively constant following treatment, the proportion of B cells was decreased in 4/9 patients.

FIG. 26: Analysis of the total cell numbers of B and myeloid cells within the peripheral blood post-treatment showed clearly increased levels of NK (5/9 patients), NKT (4/9 patients) and macrophage (3/9 patients) cell numbers post-treatment. B cell numbers showed no distinct trend with 2/9 patients showing increased levels; 4/9 patients showing no change and 3/9 patients showing decreased levels.

FIG. 27: The major changes seen post-LHRH agonist treatment was within the T cell population of the peripheral blood. In particular there was a selective increase in the proportion of naïve (CD45RA+) CD4⁺ cells, with the ratio of naïve (CD45RA⁺) to memory (CD45RO⁺) in the CD4⁺T cell subset increasing in 6/9 patients.

FIG. 28: Decrease in the impedance of skin using various laser pulse energies. There is a decrease in skin impedance in skin irradiated at energies as low as 10 mJ, using the fitted curve to interpolate data.

FIG. 29: Permeation of a pharmaceutical through skin. Permeability of the skin, using insulin as a sample pharmaceutical, was greatly increased through laser irradiation.

FIG. 30: Change in fluorescence of skin over time after the addition of 5-aminolevulenic acid (ALA) and a single impulse transient to the skin. The peak of intensity occurs at about 640 nm and is highest after 210 minutes (dashed line) post-treatment.

FIG. 31: Change in fluorescence of skin over time after the addition of 5-aminolevulenic acid (ALA) without an impulse transient. There is little change in the intensity at different time points.

FIG. 32: Comparison of change in fluorescence of skin after the addition of 5-aminolevulenic acid (ALA) and a single impulse transient under various peak stresses. The degree of permeabilization of the stratum corneum depends on the peak stress.

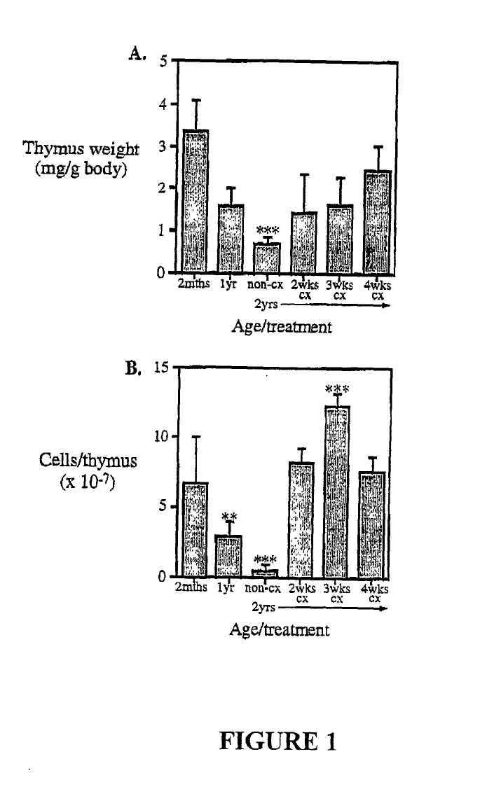

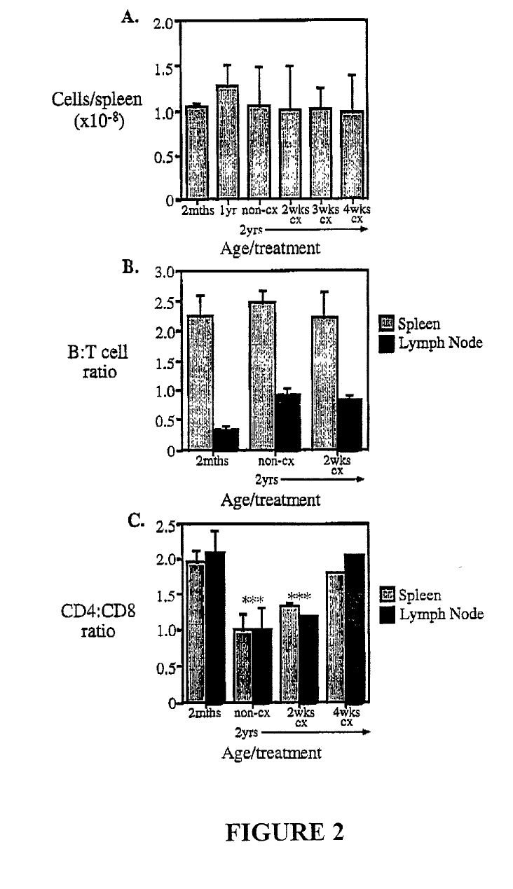

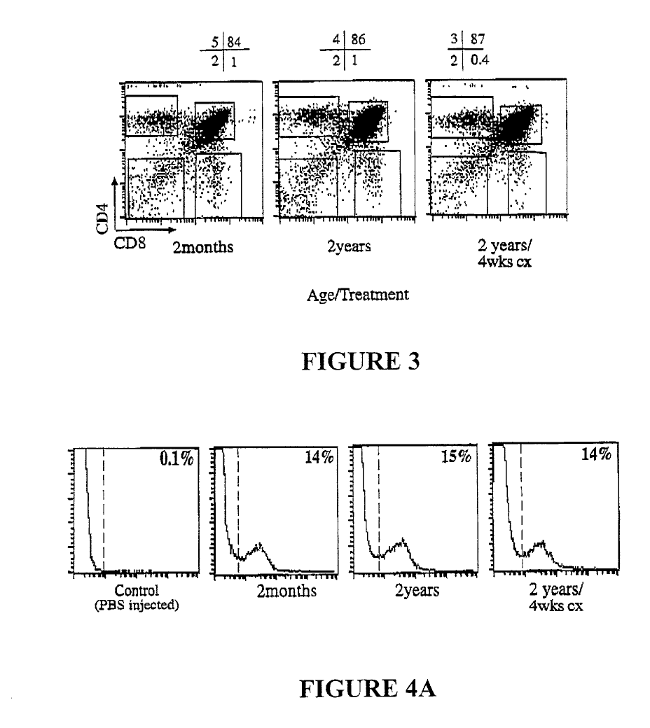

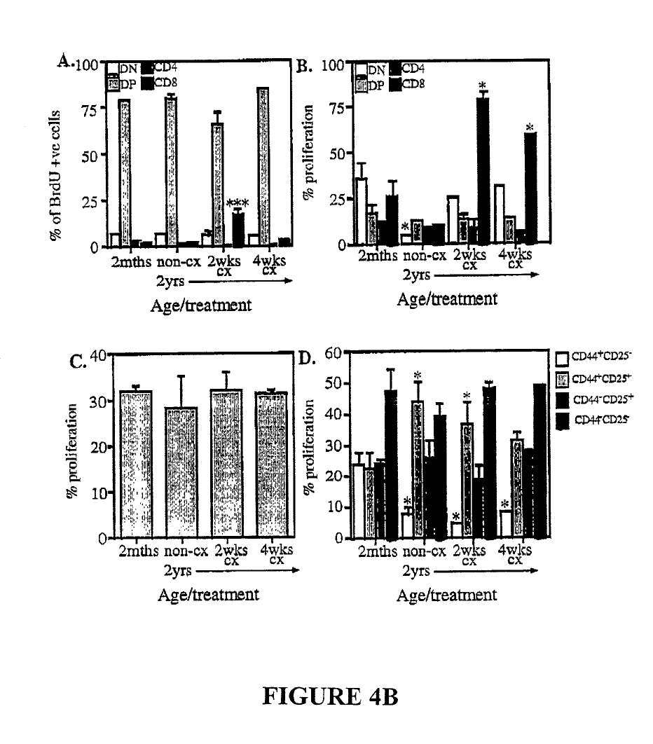

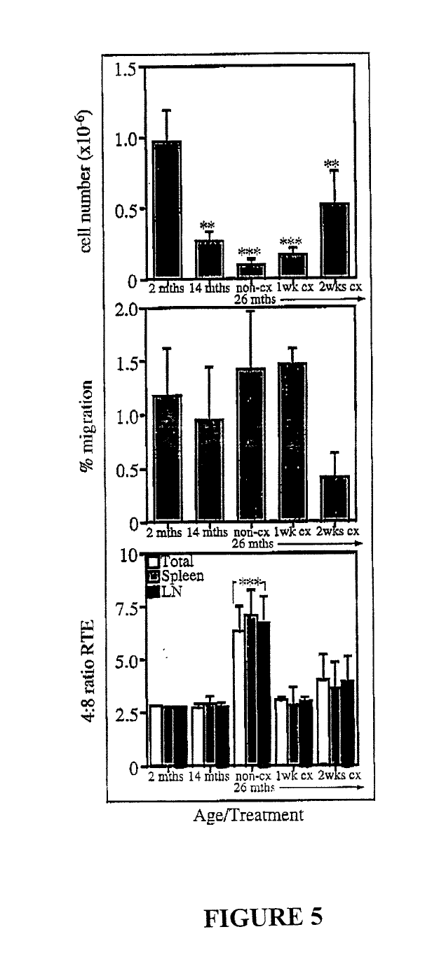

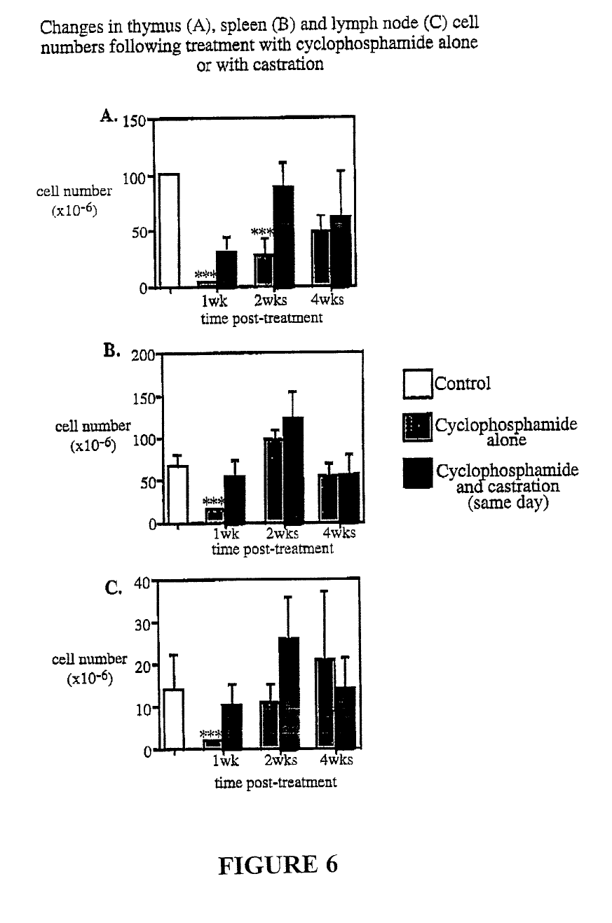

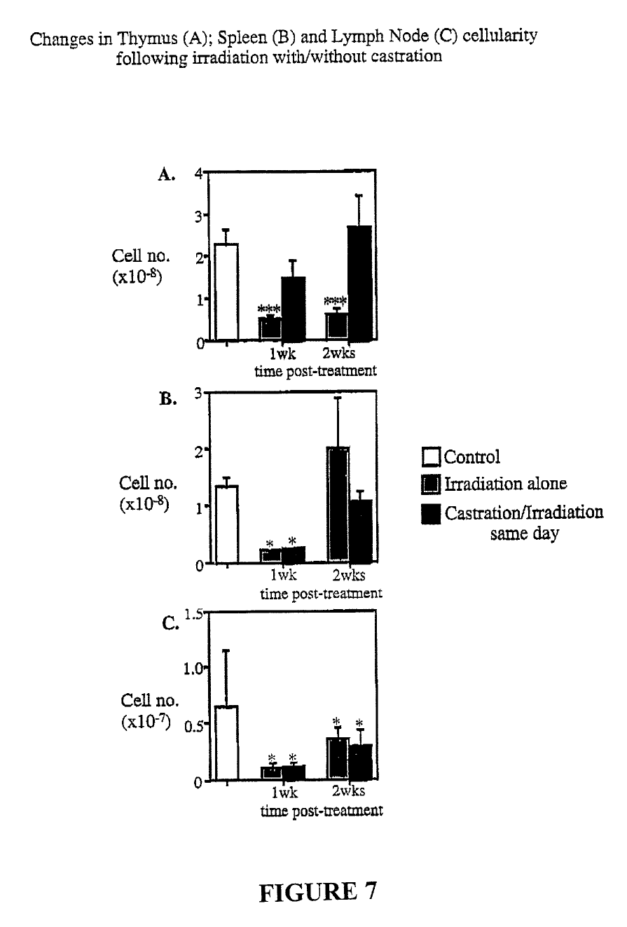

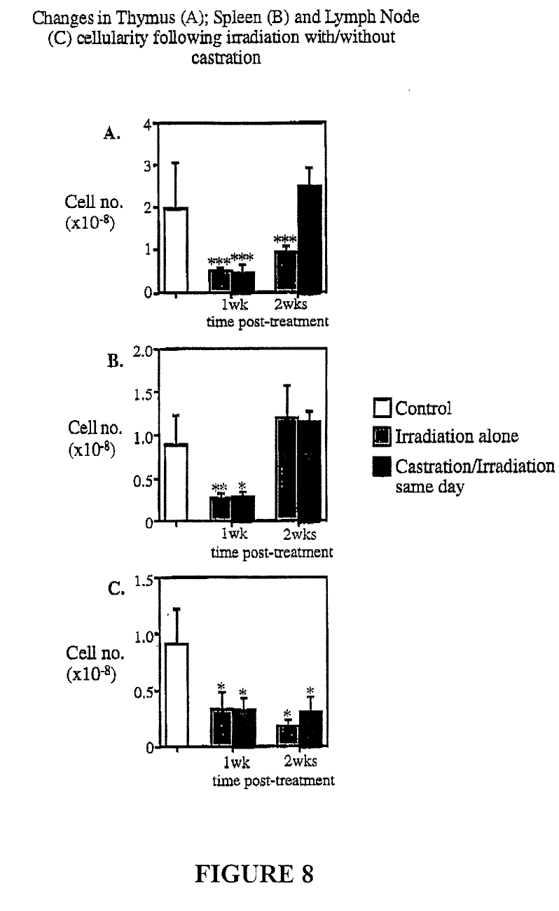

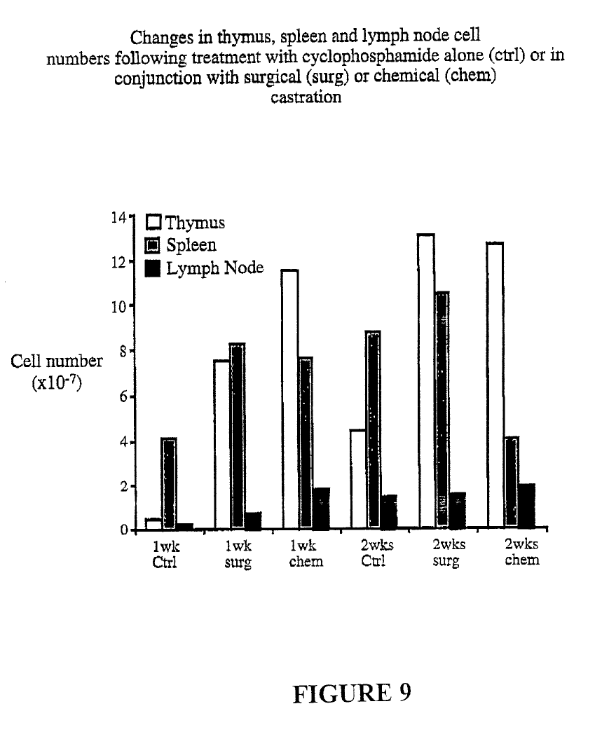

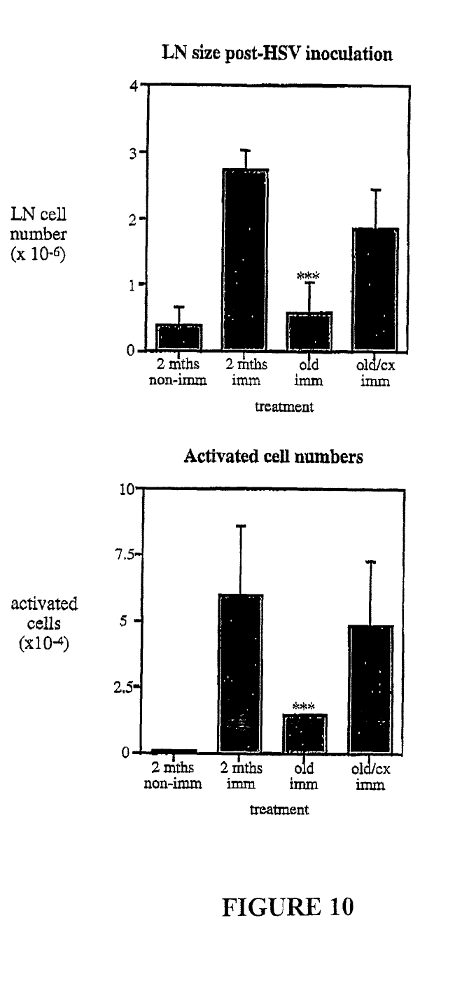

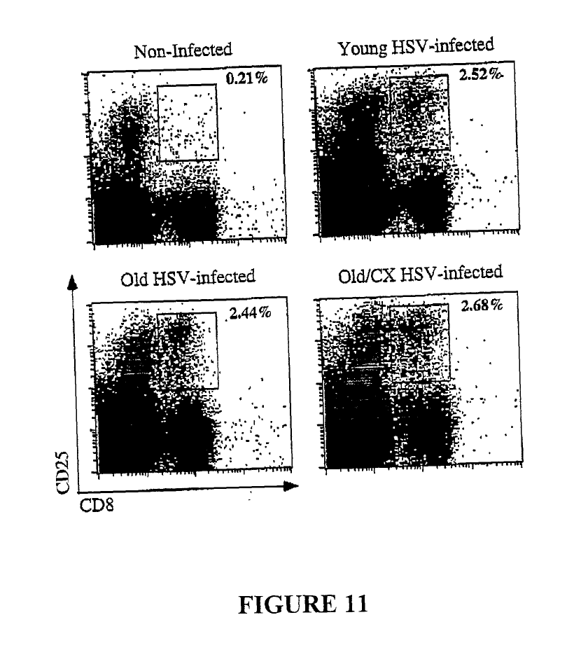

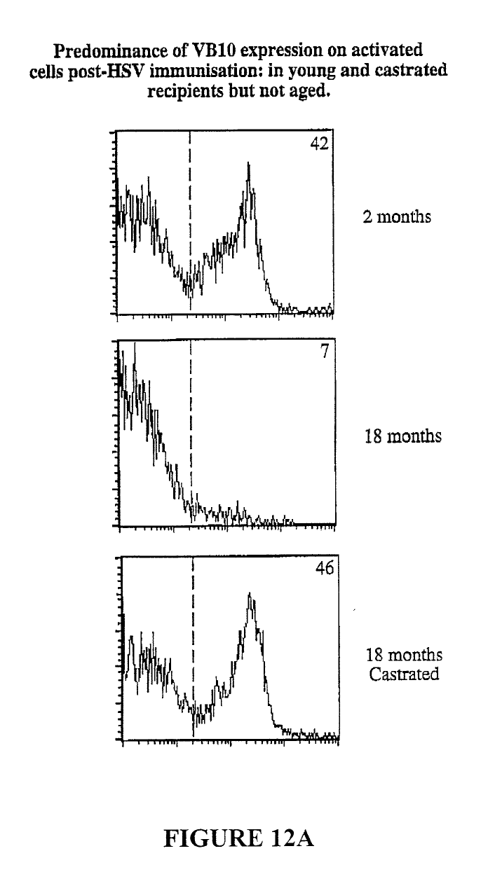

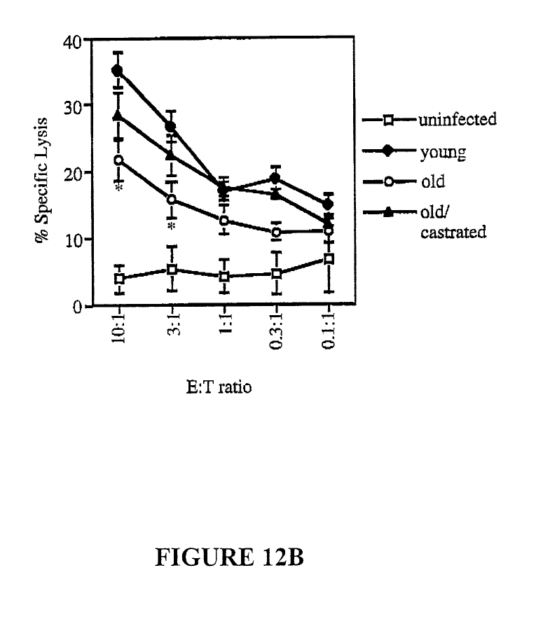

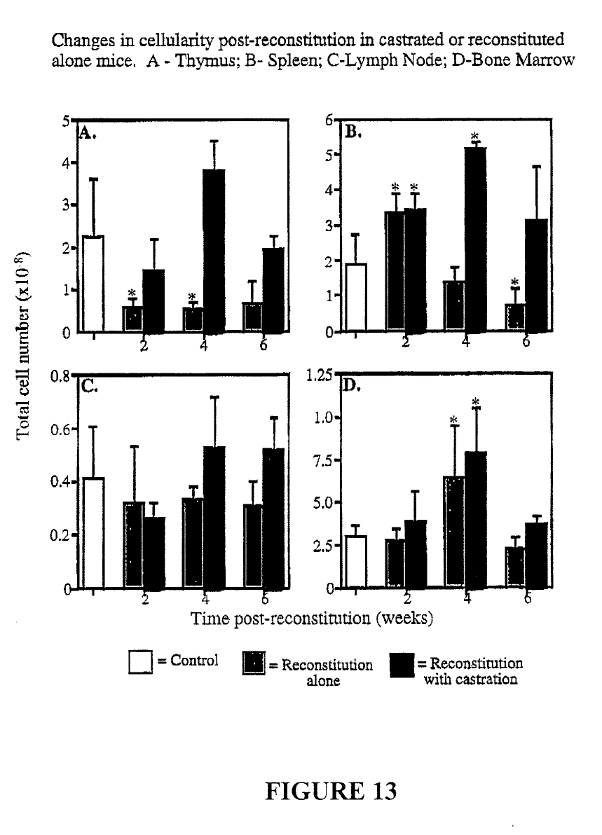

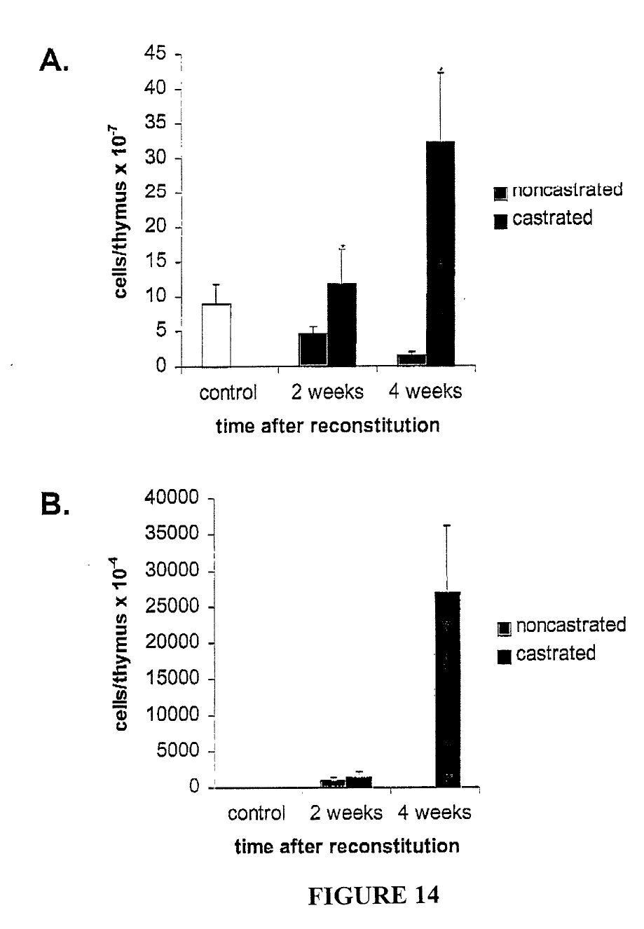

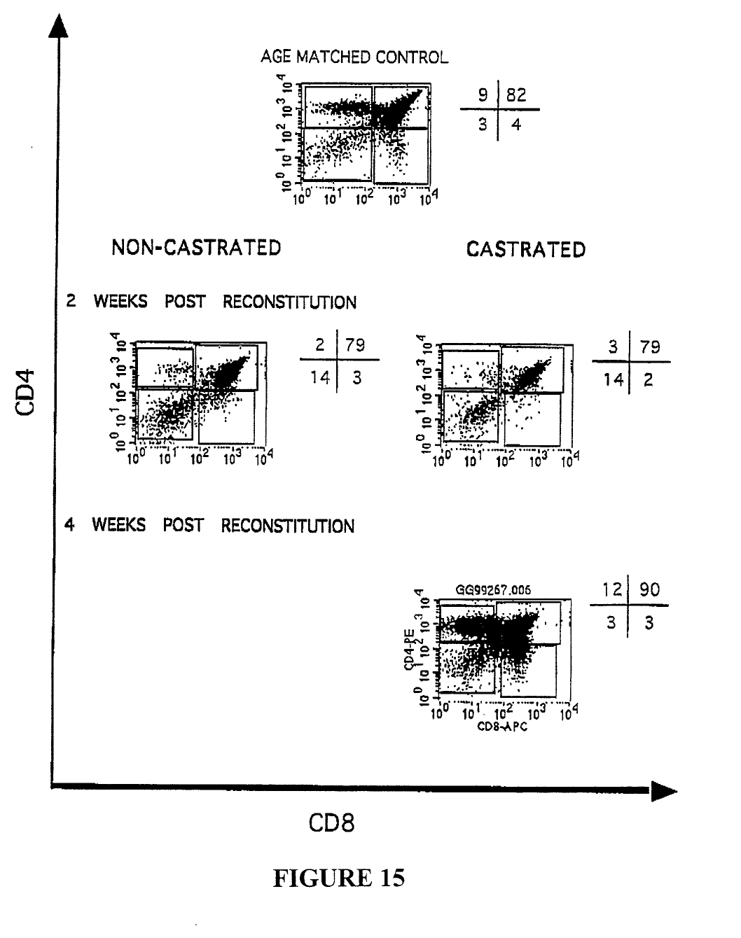

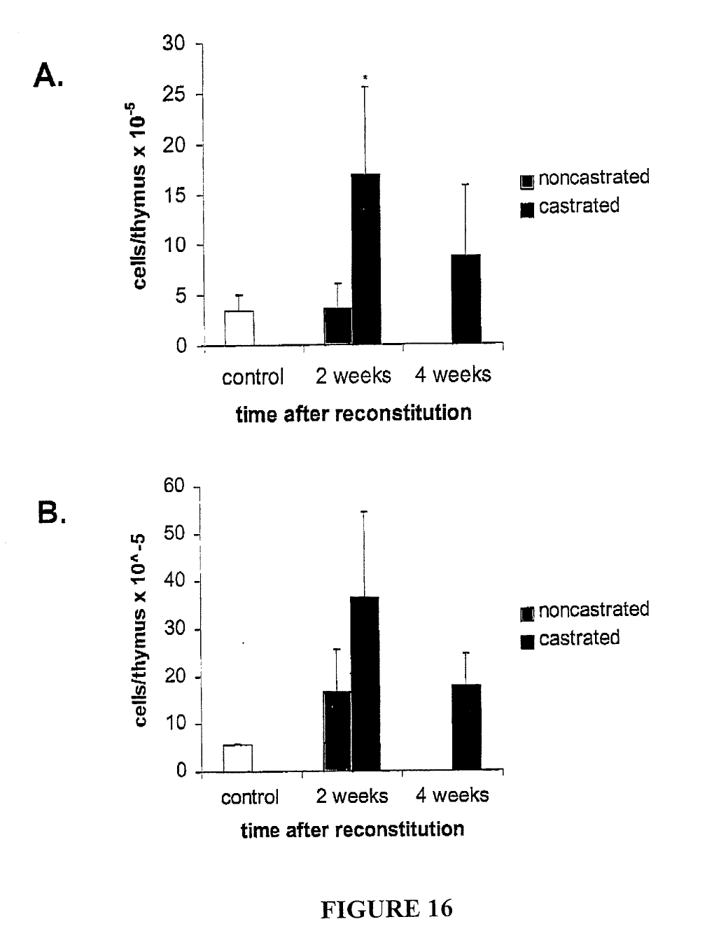

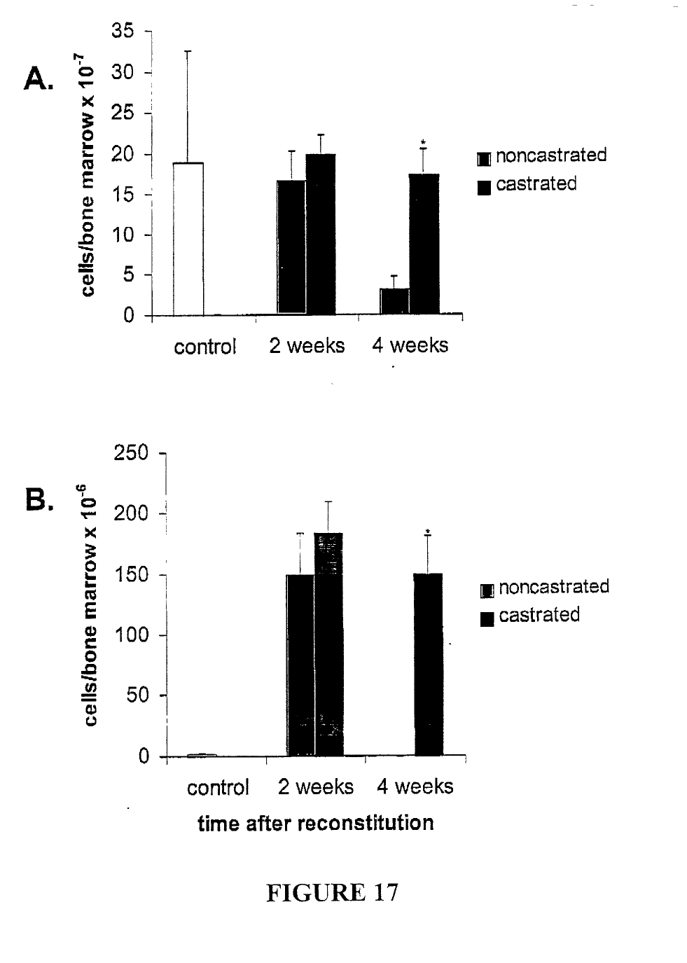

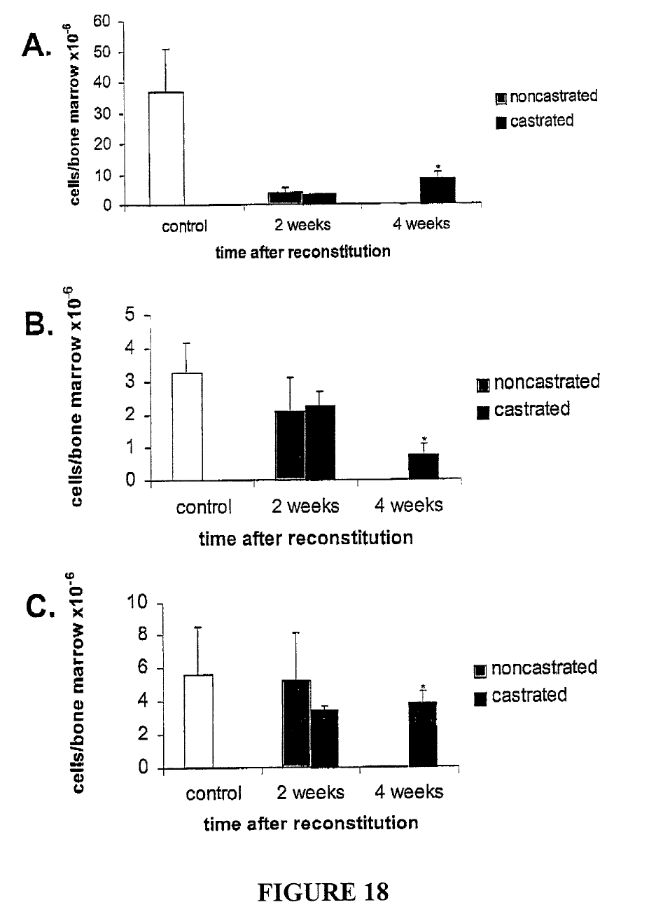

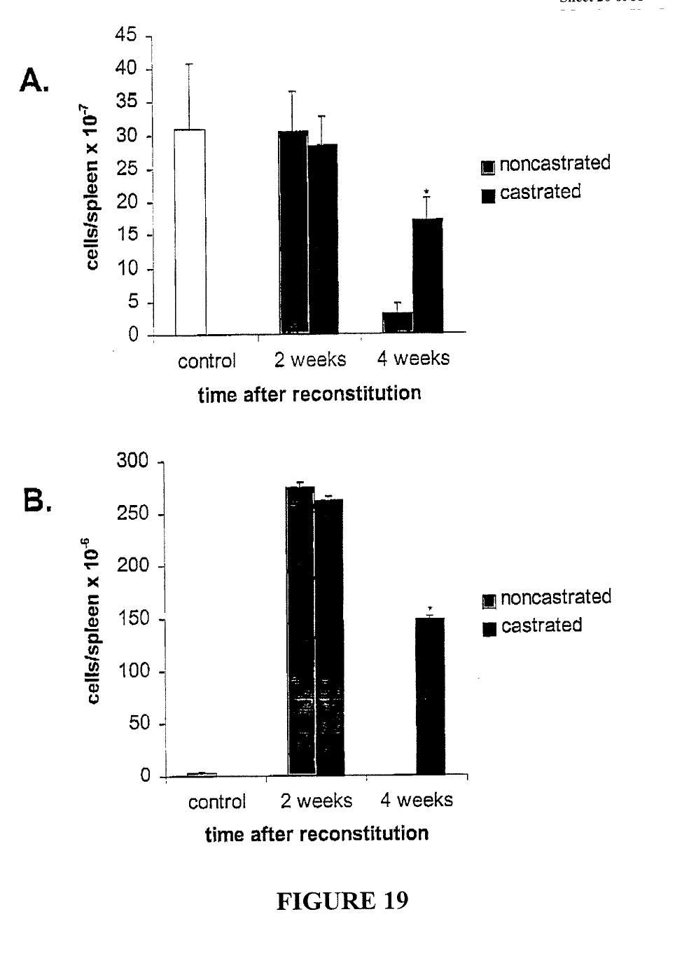

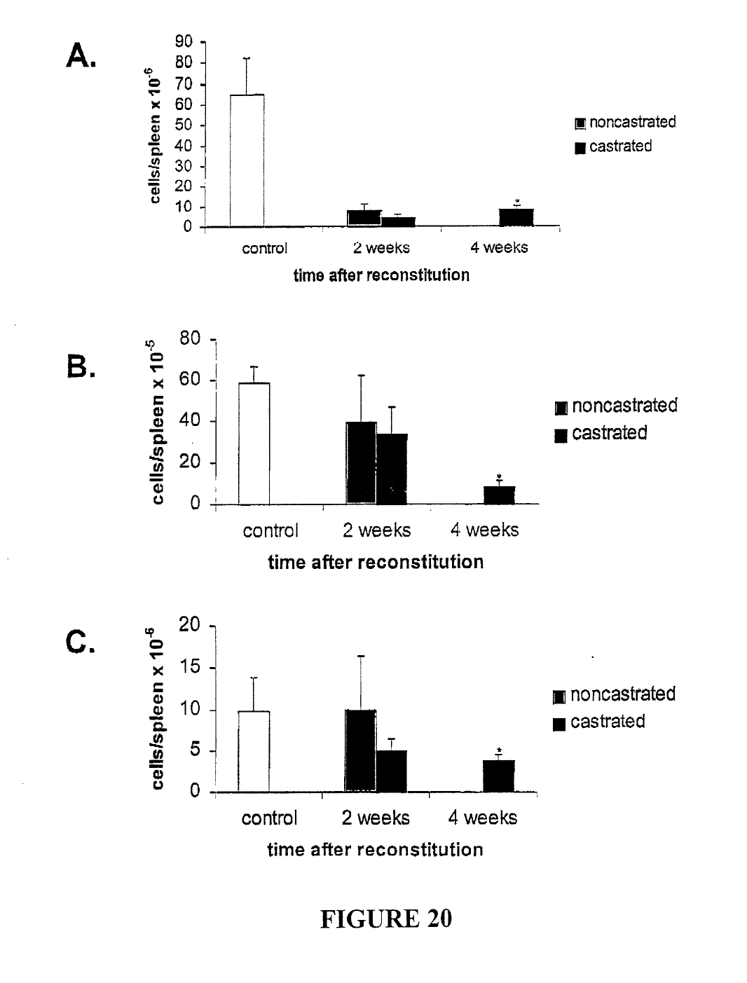

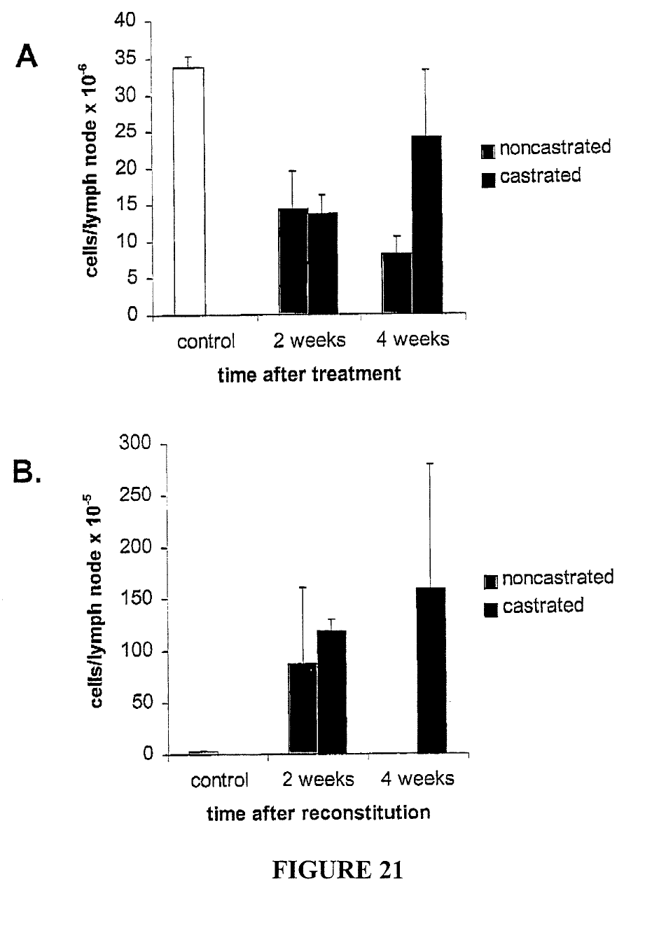

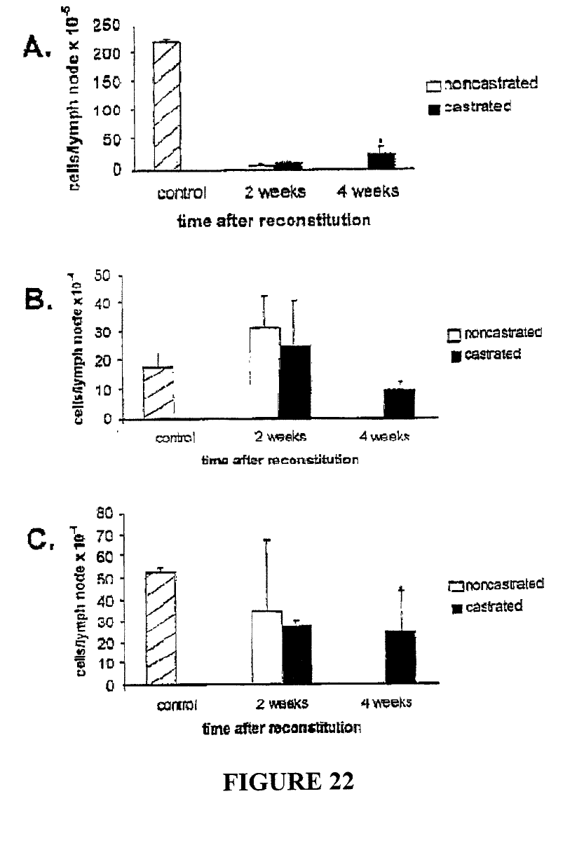

### DETAILED DESCRIPTION OF THE INVENTION

The present invention concerns methods for improving vaccine response in a patient. This is accomplished by quantitatively and qualitatively restoring the peripheral T cell pool, particularly at the level of naïve T cells. Naïve T cells are those that have not yet contacted antigen and therefore have broad based specificity, i.e., are able to respond to any one of a wide variety of antigens. As a result of the procedures of this invention a large pool of naïve T cells becomes available to respond to antigen administered in a vaccine.

In a preferred embodiment, blocking sex steroid signaling to the thymus creates this pool of naïve T cells by reactivating the atrophied thymus. This disruption reverses the hormonal status of the recipient. A preferred method for creating disruption is through castration. Methods for castration include but are not limited to chemical castration and surgical castration.

A preferred method of reactivating the thymus is by blocking the stimulatory effects of LHRH on the pituitary, which leads to a loss of the gonadotrophins FSH and LH. These gonadotrophins normally act on the gonads to release sex hormones, in particular estrogens in females and testosterone in males; the release is blocked by the loss of FSH and LH. The direct consequences of this are an immediate drop in the plasma levels of sex steroids, and as a result, progressive release of the inhibitory signals on the thymus. The degree and kinetics of thymic regrowth can be enhanced by injection of CD34⁺ hematopoietic cells (ideally autologous or syngeneic).

This invention may be used with any animal species (including humans) having sex steroid driven maturation and an immune system, such as mammals and marsupials, preferably large mammals, and most preferably humans.

The terms “regeneration,” “reactivation” and “reconstitution” and their derivatives are used interchangeably herein, and refer to the recovery of an atrophied thymus to its active state. “Castration,” as used herein, means the marked reduction or elimination of sex steroid production and distribution in the body. This effectively returns the patient to pre-pubertal status when the thymus is fully functioning. Surgical castration removes the patient's gonads.

A less permanent version of castration is through the administration of a chemical for a period of time, referred to herein as “chemical castration.” A variety of chemicals are capable of functioning in this manner. During the chemical delivery, and for a period of time afterwards, the patient's hormone production is turned off. Preferably the castration is reversed upon termination of chemical delivery.

This invention is useful in a number of conditions. For example, over a long period of time an immune system will essentially run out of naïve cells. As the number of these cells dwindles, the response to antigen presented as a vaccine also falls, as there are insufficient numbers of naïve cells available to create the response. Reactivating the thymus creates a new, large pool of naïve T cells capable of responding appropriately to the vaccine.

Additionally, a patient who has a disease or condition, such as cancer, that has induced a prolonged immune response may have antigen-specific “clonal burnout.” While the patient's naïve T cells initially responded appropriately to the foreign antigens on the tumor, thereby producing a variety of clones of cells that continue to make antibodies specific to the foreign antigens, after a long period of time these clones will lose their production capacity. In a patient with an atrophic thymus, the pool of naïve T cells available to continue the immune response is very likely to be vastly reduced and almost non-existent, and the immune reaction essentially dies out, or functions at a level that is incapable of ridding the patient of the tumor. Reactivating the thymus can prevent or relieve clonal burnout by creating a large pool of naïve T cells capable of responding to the foreign tumor antigens.

### DISRUPTION OF SEX STEROID SIGNALING TO THE THYMUS

As will be readily understood, sex steroid signaling to the thymus can be disrupted in a range of ways well known to those of skill in the art, some of which are described herein. For example, inhibition of sex steroid production or blocking of one or more sex steroid receptors within the thymus will accomplish the desired disruption, as will administration of sex steroid agonists or antagonists, or active (antigen) or passive (antibody) anti-sex steroid vaccinations. Inhibition of sex steroid production can also be achieved by administration of one or more sex steroid analogs. In some clinical cases, permanent removal of the gonads via physical castration may be appropriate.

In a preferred embodiment the sex steroid signaling to the thymus is disrupted by administration of a sex steroid analog, preferably an analog of luteinizing hormone-releasing hormone (LHRH). Sex steroid analogs and their use in therapies and chemical castration are well known. Such analogs include, but are not limited to, Eulexin (described in FR7923545, WO86/01105 and PT100899), Goserelin (described in U.S. Pat. No. 4,100,274, U.S. Pat. No. 4,128,638, GB9112859 and GB9112825), Leuprolide (described in U.S. Pat. No. 4,490,291, U.S. Pat. No. 3,972,859, U.S. Pat. No. 4,008,209, U.S. Pat. No. 4,005,063, DE2509783 and U.S. Pat. No. 4,992,421), Dioxalan derivatives such as are described in EP 413209, Triptorelin (described in U.S. Pat. No. 4,010,125, U.S. Pat. No. 4,018,726, U.S. Pat. No. 4,024,121, EP 364819 and U.S. Pat. No. 5,258,492), Meterelin (described in EP 23904), Buserelin (described in U.S. Pat. No. 4,003,884, U.S. Pat. No. 4,118,483 and U.S. Pat. No. 4,275,001), Histrelin (described in EP217659), Nafarelin (described in U.S. Pat. No. 4,234,571, WO93/15722 and EP52510), Lutrelin (described in U.S. Pat. No. 4,089,946), Leuprorelin (described in Plosker et al.) and LHRH analogs such as are described in EP181236, U.S. Pat. No. 4,608,251, U.S. Pat. No. 4,656,247, U.S. Pat. No. 4,642,332, U.S. Pat. No. 4,010,149, U.S. Pat. No. 3,992,365 and U.S. Pat. No. 4,010,149. The disclosures of each the references referred to above are incorporated herein by reference. It is currently preferred that the analog is Deslorelin (described in U.S. Pat. No. 4,218,439).

### DELIVERY OF AGENTS FOR CHEMICAL CASTRATION

Delivery of the compounds of this invention can be accomplished via a number of methods known to persons skilled in the art. One standard procedure for administering chemical inhibitors to inhibit sex steroid signaling to the thymus utilizes a single dose of an LHRH agonist that is effective for three months. For this a simple one-time i.v. or i.m. injection would not be sufficient as the agonist would be cleared from the patient's body well before the three months are over. Instead, a depot injection or an implant may be used, or any other means of delivery of the inhibitor that will allow slow release of the inhibitor. Likewise, a method for increasing the half life of the inhibitor within the body, such as by modification of the chemical, while retaining the function required herein, may be used.

Examples of more useful delivery mechanisms include, but are not limited to, laser irradiation of the skin, and creation of high pressure impulse transients (also called stress waves or impulse transients) on the skin, each method accompanied or followed by placement of the compound(s) with or without carrier at the same locus. A preferred method of this placement is in a patch placed and maintained on the skin for the duration of the treatment.

One means of delivery utilizes a laser beam, specifically focused, and lasing at an appropriate wavelength, to create small perforations or alterations in the skin of a patient. See U.S. Pat. No. 4,775,361, U.S. Pat. No. 5,643,252, U.S. Pat. No. 5,839,446, and U.S. Pat. No. 6,056,738, all of which are incorporated herein by reference. In a preferred embodiment, the laser beam has a wavelength between 0.2 and 10 microns. More preferably, the wavelength is between about 1.5 and 3.0 microns. Most preferably the wavelength is about 2.94 microns. In one embodiment, the laser beam is focused with a lens to produce an irradiation spot on the skin through the epidermis of the skin. In an additional embodiment, the laser beam is focused to create an irradiation spot only through the stratum corneum of the skin.

Several factors may be considered in defining the laser beam, including wavelength, energy fluence, pulse temporal width and irradiation spot-size. In a preferred embodiment, the energy fluence is in the range of 0.03-100,000 J/cm². More preferably, the energy fluence is in the range of 0.03-9.6 J/cm². The beam wavelength is dependent in part on the laser material, such as Er:YAG. The pulse temporal width is a consequence of the pulse width produced by, for example, a bank of capacitors, the flashlamp, and the laser rod material. The pulse width is optimally between 1 fs (femtosecond) and 1,000 μs.

According to this method the perforation or alteration produced by the laser need not be produced with a single pulse from the laser. In a preferred embodiment a perforation or alteration through the stratum corneum is produced by using multiple laser pulses, each of which perforates or alters only a fraction of the target tissue thickness.

To this end, one can roughly estimate the energy required to perforate or alter the stratum corneum with multiple pulses by taking the energy in a single pulse and dividing by the number of pulses desirable. For example, if a spot of a particular size requires 1 J of energy to produce a perforation or alteration through the entire stratum corneum, then one can produce qualitatively similar perforation or alteration using ten pulses, each having 1/10th the energy. Because it is desirable that the patient not move the target tissue during the irradiation (human reaction times are on the order of 100 ms or so), and that the heat produced during each pulse not significantly diffuse, in a preferred embodiment the pulse repetition rate from the laser should be such that complete perforation is produced in a time of less than 100 ms. Alternatively, the orientation of the target tissue and the laser can be mechanically fixed so that changes in the target location do not occur during the longer irradiation time.

To penetrate the skin in a manner that induces little or no blood flow, skin is perforated or altered through the outer surface, such as the stratum corneum layer, but not as deep as the capillary layer. The laser beam is focussed precisely on the skin, creating a beam diameter at the skin in the range of approximately 0.5 microns-5.0 cm. Optionally, the spot can be slit-shaped, with a width of about 0.05-0.5 mm and a length of up to 2.5 mm. The width can be of any size, being controlled by the anatomy of the area irradiated and the desired permeation rate of the fluid to be removed or the pharmaceutical to be applied. The focal length of the focusing lens can be of any length, but in one embodiment it is 30 mm.

By modifying wavelength, pulse length, energy fluence (which is a function of the laser energy output (in Joules) and size of the beam at the focal point (cm²)), and irradiation spot size, it is possible to vary the effect on the stratum corneum between ablation (perforation) and non-ablative modification (alteration). Both ablation and non-ablative alteration of the stratum corneum result in enhanced permeation of subsequently applied pharmaceuticals.

For example, by reducing the pulse energy while holding other variables constant, it is possible to change between ablative and non-ablative tissue-effect. Using an Er:YAG laser having a pulse length of about 300 μs, with a single pulse or radiant energy and irradiating a 2 mm spot on the skin, a pulse energy above approximately 100 mJ causes partial or complete ablation, while any pulse energy below approximately 100 mJ causes partial ablation or non-ablative alteration to the stratum corneum. Optionally, by using multiple pulses, the threshold pulse energy required to enhance permeation of body fluids or for pharmaceutical delivery is reduced by a factor approximately equal to the number of pulses.

Alternatively, by reducing the spot size while holding other variables constant, it is also possible to change between ablative and non-ablative tissue-effect. For example, halving the spot area will result in halving the energy required to produce the same effect. Irradiation down to 0.5 microns can be obtained, for example, by coupling the radiant output of the laser into the objective lens of a microscope objective. (e.g., as available from Nikon, Inc., Melville, N.Y.). In such a case, it is possible to focus the beam down to spots on the order of the limit of resolution of the microscope, which is perhaps on the order of about 0.5 microns. In fact, if the beam profile is Gaussian, the size of the affected irradiated area can be less than the measured beam size and can exceed the imaging resolution of the microscope. To non-ablatively alter tissue in this case, it would be suitable to use a 3.2 J/cm² energy fluence, which for a half-micron spot size would require a pulse energy of about 5 nJ. This low a pulse energy is readily available from diode lasers, and can also be obtained from, for example, the Er:YAG laser by attenuating the beam by an absorbing filter, such as glass.

Optionally, by changing the wavelength of radiant energy while holding the other variables constant, it is possible to change between an ablative and non-ablative tissue-effect. For example, using Ho:YAG (holmium: YAG; 2.127 microns) in place of the Er:YAG (erbium:YAG;2.94 microns) laser, would result in less absorption of energy by the tissue, creating less of a perforation or alteration.

Picosecond and femtosecond pulses produced by lasers can also be used to produce alteration or ablation in skin. This can be accomplished with modulated diode or related microchip lasers, which deliver single pulses with temporal widths in the 1 femtosecond to 1 ms range. (See D. Stern et al., “Corneal Ablation by Nanosecond, Picosecond, and Femtosecond Lasers at 532 and 625 nm,” Corneal Laser Ablation, Vol. 107, pp. 587-592 (1989), incorporated herein by reference, which discloses the use of pulse lengths down to 1 femtosecond).

Another delivery method uses high pressure impulse transients on skin to create permeability. See U.S. Pat. No. 5,614,502, and U.S. Pat. No. 5,658,892, both of which are incorporated herein by reference. High pressure impulse transients, e.g., stress waves (e.g., laser stress waves (LSW) when generated by a laser), with specific rise times and peak stresses (or pressures), can safely and efficiently effect the transport of compounds, such as those of the present invention, through layers of epithelial tissues, such as the stratum corneum and mucosal membranes. These methods can be used to deliver compounds of a wide range of sizes regardless of their net charge. In addition, impulse transients used in the present methods avoid tissue injury.

Prior to exposure to an impulse transient, an epithelial tissue layer, e.g., the stratum corneum, is likely impermeable to a foreign compound; this prevents diffusion of the compound into cells underlying the epithelial layer. Exposure of the epithelial layer to the impulse transients enables the compound to diffuse through the epithelial layer. The rate of diffusion, in general, is dictated by the nature of the impulse transients and the size of the compound to be delivered.

The rate of penetration through specific epithelial tissue layers, such as the stratum corneum of the skin, also depends on several other factors including pH, the metabolism of the cutaneous substrate tissue, pressure differences between the region external to the stratum corneum, and the region internal to the stratum corneum, as well as the anatomical site and physical condition of the skin. In turn, the physical condition of the skin depends on health, age, sex, race, skin care, and history. For example, prior contacts with organic solvents or surfactants affect the physical condition of the skin.

The amount of compound delivered through the epithelial tissue layer will also depend on the length of time the epithelial layer remains permeable, and the size of the surface area of the epithelial layer which is made permeable. The properties and characteristics of impulse transients are controlled by the energy source used to create them. See WO 98/23325, which is incorporated herein by reference. However, their characteristics are modified by the linear and non-linear properties of the coupling medium through which they propagate. The linear attenuation caused by the coupling medium attenuates predominantly the high frequency components of the impulse transients. This causes the bandwidth to decrease with a corresponding increase in the rise time of the impulse transient. The non-linear properties of the coupling medium, on the other hand, cause the rise time to decrease. The decrease of the rise time is the result of the dependence of the sound and particle velocity on stress (pressure). As the stress increases, the sound and the particle velocity increase as well. This causes the leading edge of the impulse transient to become steeper. The relative strengths of the linear attenuation, non-linear coefficient, and the peak stress determine how long the wave has to travel for the increase in steepness of rise time to become substantial.

The rise time, magnitude, and duration of the impulse transient are chosen to create a nondestructive (i.e., non-shock wave) impulse transient that temporarily increases the permeability of the epithelial tissue layer. Generally the rise time is at least 1 ns, and is more preferably about 10 ns.

The peak stress or pressure of the impulse transients varies for different epithelial tissue or cell layers. For example, to transport compounds through the stratum corneum, the peak stress or pressure of the impulse transient should be set to at least 400 bar; more preferably at least 1,000 bar, but no more than about 2,000 bar.

For epithelial mucosal layers, the peak pressure should be set to between 300 bar and 800 bar, and is preferably between 300 bar and 600 bar.

The impulse transients preferably have durations on the order of a few tens of ns, and thus interact with the epithelial tissue for only a short period of time.

Following interaction with the impulse transient, the epithelial tissue is not permanently damaged, but remains permeable for up to about three minutes.

In addition, the new methods involve the application of only a few discrete high amplitude pulses to the patient. The number of impulse transients administered to the patient is typically less than 100, more preferably less than 50, and most preferably less than 10. When multiple optical pulses are used to generate the impulse transient, the time duration between sequential pulses is 10 to 120 seconds, which is long enough to prevent permanent damage to the epithelial tissue.

Properties of impulse transients can be measured using methods standard in the art. For example, peak stress or pressure, and rise time can be measured using a polyvinylidene fluoride (PVDF) transducer method as described in Doukas et al., Ultrasound Med. Biol., 21:961 (1995).

Impulse transients can be generated by various energy sources. The physical phenomenon responsible for launching the impulse transient is, in general, chosen from three different mechanisms: (1) thermoelastic generation; (2) optical breakdown; or (3) ablation.

For example, the impulse transients can be initiated by applying a high energy laser source to ablate a target material, and the impulse transient is then coupled to an epithelial tissue or cell layer by a coupling medium. The coupling medium can be, for example, a liquid or a gel, as long as it is non-linear. Thus, water, oil such as castor oil, an isotonic medium such as phosphate buffered saline (PBS), or a gel such as a collagenous gel, can be used as the coupling medium.

In addition, the coupling medium can include a surfactant that enhances transport, e.g., by prolonging the period of time in which the stratum corneum remains permeable to the compound following the generation of an impulse transient. The surfactant can be, e.g., ionic detergents or nonionic detergents and thus can include, e.g., sodium lauryl sulfate, cetyl trimethyl ammonium bromide, and lauryl dimethyl amine oxide.

The absorbing target material acts as an optically triggered transducer. Following absorption of light, the target material undergoes rapid thermal expansion, or is ablated, to launch an impulse transient. Typically, metal and polymer films have high absorption coefficients in the visible and ultraviolet spectral regions.

Many types of materials can be used as the target material in conjunction with a laser beam, provided they fully absorb light at the wavelength of the laser used. The target material can be composed of a metal such as aluminum or copper; a plastic, such as polystyrene, e.g., black polystyrene; a ceramic; or a highly concentrated dye solution. The target material must have dimensions larger than the cross-sectional area of the applied laser energy. In addition, the target material must be thicker than the optical penetration depth so that no light strikes the surface of the skin. The target material must also be sufficiently thick to provide mechanical support. When the target material is made of a metal, the typical thickness will be 1/32 to 1/16 inch. For plastic target materials, the thickness will be 1/16 to ⅛ inch.

Impulse transients can be also enhanced using confined ablation. In confined ablation, a laser beam transparent material, such as a quartz optical window, is placed in close contact with the target material. Confinement of the plasma, created by ablating the target material by using the transparent material, increases the coupling coefficient by an order of magnitude (Fabro et al., J. Appl. Phys., 68:775, 1990). The transparent material can be quartz, glass, or transparent plastic.

Since voids between the target material and the confining transparent material allow the plasma to expand, and thus decrease the momentum imparted to the target, the transparent material is preferably bonded to the target material using an initially liquid adhesive, such as carbon-containing epoxies, to prevent such voids.

The laser beam can be generated by standard optical modulation techniques known in the art, such as by employing Q-switched or mode-locked lasers using, for example, electro- or acousto-optic devices. Standard commercially available lasers that can operate in a pulsed mode in the infrared, visible, and/or infrared spectrum include Nd:YAG, Nd:YLF, CO₂, excimer, dye, Ti:sapphire, diode, holmium (and other rare-earth materials), and metal-vapor lasers. The pulse widths of these light sources are adjustable, and can vary from several tens of picoseconds (ps) to several hundred microseconds. For use in the new methods, the optical pulse width can vary from 100 ps to about 200 ns and is preferably between about 500 ps and 40 ns.

Impulse transients can also be generated by extracorporeal lithotripters (one example is described in Coleman et al., Ultrasound Med. Biol., 15:213-227, 1989). These impulse transients have rise times of 30 to 450 ns, which is longer than laser-generated impulse transients. To form an impulse transient of the appropriate rise time for the new methods using an extracorporeal lithotripter, the impulse transient is propagated in a non-linear coupling medium (e.g., water) for a distance determined by equation (1), above. For example, when using a lithotripter creating an impulse transient having a rise time of 100 ns and a peak pressure of 500 barr, the distance that the impulse transient should travel through the coupling medium before contacting an epithelial cell layer is approximately 5 mm.

An additional advantage of this approach for shaping impulse transients generated by lithotripters is that the tensile component of the wave will be broadened and attenuated as a result of propagating through the non-linear coupling medium. This propagation distance should be adjusted to produce an impulse transient having a tensile component that has a pressure of only about 5 to 10% of the peak pressure of the compressive component of the wave. Thus, the shaped impulse transient will not damage tissue.

The type of lithotripter used is not critical. Either an electrohydraulic, electromagnetic, or piezoelectric lithotripter can be used.

The impulse transients can also be generated using transducers, such as piezoelectric transducers. Preferably, the transducer is in direct contact with the coupling medium, and undergoes rapid displacement following application of an optical, thermal, or electric field to generate the impulse transient. For example, dielectric breakdown can be used, and is typically induced by a high-voltage spark or piezoelectric transducer (similar to those used in certain extracorporeal lithotripters, Coleman et al., Ultrasound Med. Biol., 15:213-227, 1989). In the case of a piezoelectric transducer, the transducer undergoes rapid expansion following application of an electrical field to cause a rapid displacement in the coupling medium.

In addition, impulse transients can be generated with the aid of fiber optics. Fiber optic delivery systems are particularly maneuverable and can be used to irradiate target materials located adjacent to epithelial tissue layers to generate impulse transients in hard-to reach places. These types of delivery systems, when optically coupled to lasers, are preferred as they can be integrated into catheters and related flexible devices, and used to irradiate most organs in the human body. In addition, to launch an impulse transient having the desired rise times and peak stress, the wavelength of the optical source can be easily tailored to generate the appropriate absorption in a particular target material.

Alternatively, an energetic material can produce an impulse transient in response to a detonating impulse. The detonator can detonate the energetic material by causing an electrical discharge or spark.

Hydrostatic pressure can be used in conjunction with impulse transients to enhance the transport of a compound through the epithelial tissue layer. Since the effects induced by the impulse transients last for several minutes, the transport rate of a drug diffusing passively through the epithelial cell layer along its concentration gradient can be increased by applying hydrostatic pressure on the surface of the epithelial tissue layer, e.g., the stratum corneum of the skin, following application of the impulse transient.

### IMPROVEMENT OF VACCINE RESPONSE

By the methods described herein, the sex steroid-induced atrophic thymus is dramatically restored structurally and functionally to approximately its optimal pre-pubertal capacity in all currently definable terms. This includes the number, type and proportion of all T cell subsets. Also included are the complex stromal cells and their three dimensional architecture which constitute the thymic microenvironment required for producing T cells. The newly generated T cells emigrate from the thymus and restore peripheral T cell levels and function.

The reactivation of the thymus can be supplemented by the addition of CD34⁺ hematopoietic stem cells (HSC) slightly before or at the time the thymus begins to regenerate. Ideally these cells are autologous or syngeneic and have been obtained from the patient or twin prior to thymus reactivation. The cells can be obtained by sorting CD34⁺ cells from the patient's blood and/or bone marrow. The number of HSC can be enhanced in several ways, including (but not limited to) by administering G-CSF (Neupogen, Amgen) to the patient prior to collecting cells, culturing the collected cells in Stem Cell Growth Factor, and/or administering G-CSF to the patient after CD34⁺ cell supplementation. Alternatively, the CD34⁺ cells need not be sorted from the blood or BM if their population is enhanced by prior injection of G-CSF into the patient.

Within 3-4 weeks of the start of blockage of sex steroid signaling (approximately 2-3 weeks after the initiation of LHRH treatment), the first new T cells are present in the blood stream. Full development of the T cell pool, however, may take 3-4 months. In principle vaccination could begin soon after the appearance of the newly produced naïve cells; however, it is preferable to wait until 4-6 weeks after the initiation of LHRH therapy to begin vaccination, when enough new T cells to create a strong response will have been produced and will have undergone any necessary post-thymic maturation.

This procedure can be combined with any other form of immune system stimulation, including adjuvant, accessory molecules, and cytokine therapies. For example, useful cytokines include but are not limited to interleukin 2 (IL2) as a general immune growth factor, IL4 to skew the response to Th2 (humoral immunity), and interferon γ to skew the response to Th1 (cell mediated, inflammatory responses). Accessory molecules include but are not limited to inhibitors of CTLA4, which enhance the general immune response by facilitating the CD28/B7.1,B7.2 stimulation pathway, which is normally inhibited by CTLA4.

### EXAMPLES

The following Examples provide specific versions of methods of the invention, and are not to be construed as limiting the invention to their content. For convenience these examples describe delivery of an LHRH agonist to block sex steroid signaling to the thymus. However, the scope of the invention is not so limited.

### Example 1

### Sex Steroid Ablation Therapy

The patient was given sex steroid ablation therapy in the form of delivery of an LHRH agonist. This was given in the form of either Leucrin (depot injection; 22.5 mg) or Zoladex (implant; 10.8 mg), either one as a single dose effective for 3 months. This was effective in reducing sex steroid levels sufficiently to reactivate the thymus. In some cases it is also necessary to deliver a suppresser of adrenal gland production of sex steroids, such as Cosudex (5 mg/day) as one tablet per day for the duration of the sex steroid ablation therapy. Adrenal gland production of sex steroids makes up around 10-15% of a human's steroids.

Reduction of sex steroids in the blood to minimal values took about 1-3 weeks; concordant with this was the reactivation of the thymus. In some cases it is necessary to extend the treatment to a second 3 month injection/implant.

### Example 2

### Alternative Delivery Method

In place of the 3 month depot or implant administration of the LHRH agonist, alternative methods can be used. In one example the patient's skin may be irradiated by a laser such as an Er:YAG laser, to ablate or alter the skin so as to reduce the impeding effect of the stratum corneum.

A. Laser Ablation or Alteration: An infrared laser radiation pulse was formed using a solid state, pulsed, Er:YAG laser consisting of two flat resonator mirrors, an Er:YAG crystal as an active medium, a power supply, and a means of focusing the laser beam. The wavelength of the laser beam was 2.94 microns. Single pulses were used.

The operating parameters were as follows: The energy per pulse was 40, 80 or 120 mJ, with the size of the beam at the focal point being 2 mm, creating an energy fluence of 1.27, 2.55 or 3.82 J/cm². The pulse temporal width was 300 μs, creating an energy fluence rate of 0.42, 0.85 or 1.27×10⁴ W/cm².

Subsequently, an amount of LHRH agonist is applied to the skin and spread over the irradiation site. The LHRH agonist may be in the form of an ointment so that it remains on the site of irradiation. Optionally, an occlusive patch is placed over the agonist in order to keep it in place over the irradiation site.

Optionally a beam splitter is employed to split the laser beam and create multiple sites of ablation or alteration. This provides a faster flow of LHRH agonist through the skin into the blood stream. The number of sites can be predetermined to allow for maintenance of the agonist within the patient's system for the requisite approximately 30 days.

B. Pressure Wave: A dose of LHRH agonist is placed on the skin in a suitable container, such as a plastic flexible washer (about 1 inch in diameter and about 1/16 inch thick), at the site where the pressure wave is to be created. The site is then covered with target material such as a black polystyrene sheet about 1 mm thick. A Q-switched solid state ruby laser (20 ns pulse duration, capable of generating up to 2 joules per pulse) is used to generate the laser beam, which hits the target material and generates a single impulse transient. The black polystyrene target completely absorbs the laser radiation so that the skin is exposed only to the impulse transient, and not laser radiation. No pain is produced from this procedure. The procedure can be repeated daily, or as often as required, to maintain the circulating blood levels of the agonist.

### Example 3

### Optional Administration Of HSC

In one embodiment hematopoietic stem cells (HSC) are given to the patient to speed the reactivation of the thymus. These are preferably autologous or syngeneic, but HSC from a mismatched donor (allogeneic or xenogeneic) can also be used. Where practical, the level of HSC in the patient's or donor's blood is enhanced by injecting the patient or donor with granulocyte-colony stimulating factor (G-CSF) at 10 μg/kg for 2-5 days prior to cell collection. CD34⁺ cells are purified from the patient's or a donor's blood or bone marrow, preferably using a flow cytometer or immunomagnetic beading. HSC are identified by flow cytometry as being CD34⁺. Optionally these HSC are expanded ex vivo with Stem Cell Factor. At approximately 1-3 weeks post LHRH agonist delivery, just before or at the time the thymus begins to regenerate, the patient is injected with the HSC, optimally at a dose of about 2-4×10⁶ cells/kg. Optionally G-CSF may also be injected into the recipient to assist in expansion of the HSC.

When the HSC are from a mismatched donor, T cell ablation and immunosuppressive therapy may be applied to the recipient to prevent rejection of the foreign HSC. In an example of such therapy, anti-T cell antibodies in the form of a daily injection of 15 mg/kg of Atgam (xeno anti-T cell globulin, Pharmacia Upjohn) are administered for a period of 10 days in combination with an inhibitor of T cell activation, cyclosporin A, 3 mg/kg, as a continuous infusion for 3-4 weeks followed by daily tablets at 9 mg/kg as needed. The prevention of T cell reactivity may also be combined with inhibitors of second level signals such as interleukins or cell adhesion molecules to enhance the T cell ablation. This treatment is begun before or at the same time as the beginning of sex steroid ablation.

The reactivated thymus takes up the purified HSC and converts them into new T cells. In the event that unmatched donor HSC are used, the donor dendritic cells will tolerize any T cells that are potentially reactive with the patient by inducing deletion by cell death, or by inducing tolerance through immunoregulatory cells.

Since the new T cells are purged of potentially self reactive and host reactive cells, having been positively selected by the host thymic epithelium, they are able to respond to normal infections by recognizing peptide presented by host APC in the periphery. Both patient and donor CD34⁺ HSC develop into dendritic cells, and subsequently into the patient's lymphoid system organs, and establish an immune system virtually identical to that of the patient alone, albeit with enhanced amounts of naïve T cells. Thus normal immunoregulatory mechanisms will be present.

### Example 4

### Regeneration Of The Thymus

The reactivated thymus takes up the HSC and converts them into new T cells, which emigrate into the blood stream and rebuild an optimal peripheral T cell pool in the patient. In particular, there is a major increase in the levels and proportions of naïve T cells, which greatly increases the number of potential responding cells in vaccination programs. The correct ratios of Th:Tc and Th1:Th2 cells ensures an optimal type of response.

When the new cohort of mature T cells have begun exiting the thymus, blood is taken from the patient and the status of T cells (and indeed all blood cells) is examined. In particular the T cells are examined for whether they are Th1 or Th2, and naïve or memory. In addition the types of cytokines they produce (Th1 versus Th2) are examined.

Immunosuppressive therapy, if used due to administration of mismatched HSC, is gradually reduced to allow defense against infection, and is stopped completely when there is no sign of rejection, as indicated in part by the presence of activated T cells in the blood. Because the HSC have a strong self-renewal capacity, the hematopoietic chimera formed will be stable, theoretically for the life of the patient, as in the situation where no mismatched HSC are used.

### Example 5

### Use Of LHRH Agonist To Reactivate The Thymus In Humans

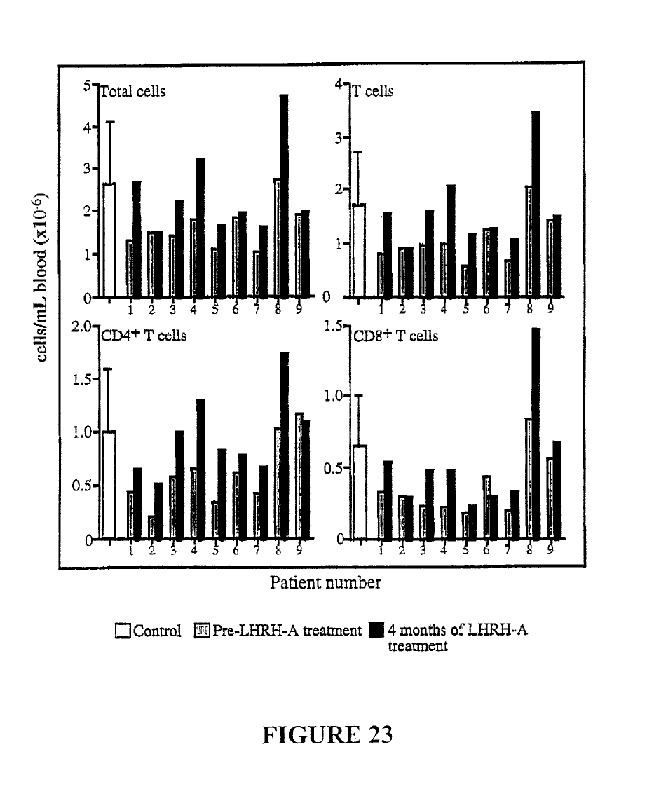

In order to show that a human thymus can be reactivated by the methods of this invention, these methods were used on patients who had been treated with chemotherapy for prostate cancer. Prostate cancer patients were evaluated before and 4 months after sex steroid ablation therapy. The results are summarized in FIGS. 23-27. Collectively the data demonstrate qualitative and quantitative improvement of the status of T cells in many patients, and the effect of LHRH therapy on total numbers of lymphocytes and T cells subsets thereof.

The phenotypic composition of peripheral blood lymphocytes was analyzed in patients (all>60 years) undergoing LHRH agonist treatment for prostate cancer (FIG. 23). Patient samples were analyzed before treatment and 4 months after beginning LHRH agonist treatment. Total lymphocyte cell numbers per ml of blood were at the lower end of control values before treatment in all patients. Following treatment, 6/9 patients showed substantial increases in total lymphocyte counts (in some cases a doubling of total cells was observed). Correlating with this was an increase in total T cell numbers in 6/9 patients. Within the CD4⁺ subset, this increase was even more pronounced with 8/9 patients demonstrating increased levels of CD4⁺ T cells. A less distinctive trend was seen within the CD8⁺ subset with 4/9 patients showing increased levels, generally to a smaller extent than CD4⁺ T cells.

The Effect Of LHRH Therapy On The Proportion Of T Cells Subsets:

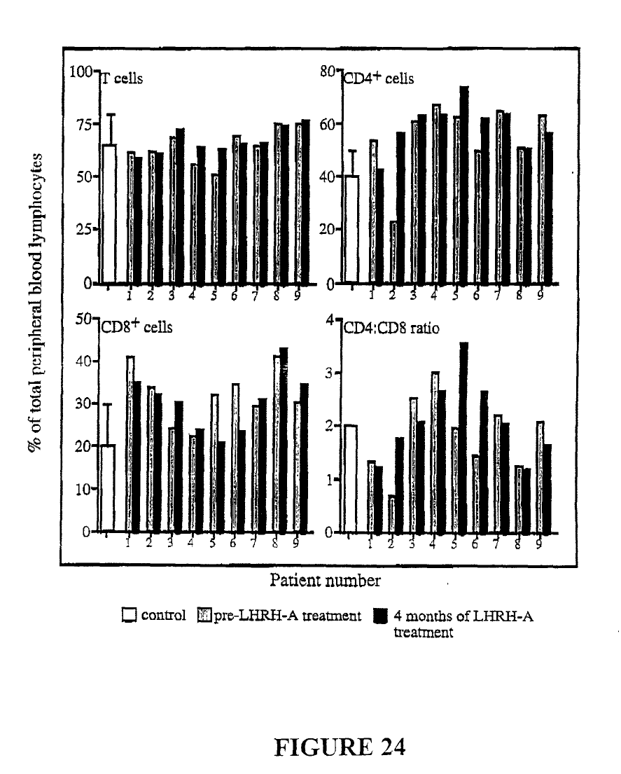

Analysis of patient blood before and after LHRH agonist treatment demonstrated no substantial changes in the overall proportion of T cells, CD4⁺ or CD8⁺ T cells and a variable change in the CD4⁺:CD8⁺ ratio following treatment (FIG. 24). This indicates that there was little effect of treatment on the homeostatic maintenance of T cell subsets despite the substantial increase in overall T cell numbers following treatment. All values were comparative to control values.

The Effect Of LHRH Therapy On The Proportion Of B Cells And Myeloid Cells:

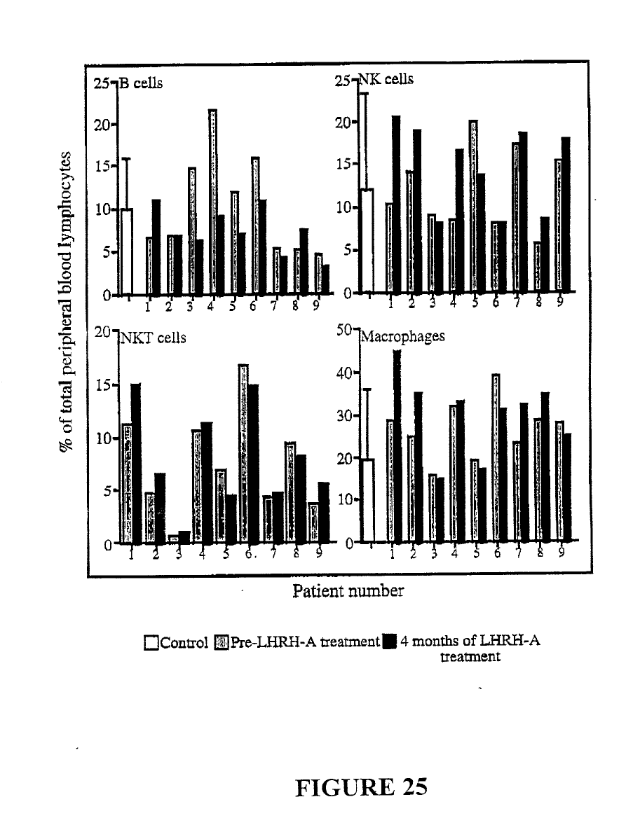

Analysis of the proportions of B cells and myeloid cells (NK, NKT and macrophages) within the peripheral blood of patients undergoing LHRH agonist treatment demonstrated a varying degree of change within subsets (FIG. 25). While NK, NKT and macrophage proportions remained relatively constant following treatment, the proportion of B cells was decreased in 4/9 patients.

The Effect Of LHRH Agonist Therapy On The Total Number Of B Cells And Myeloid Cells:

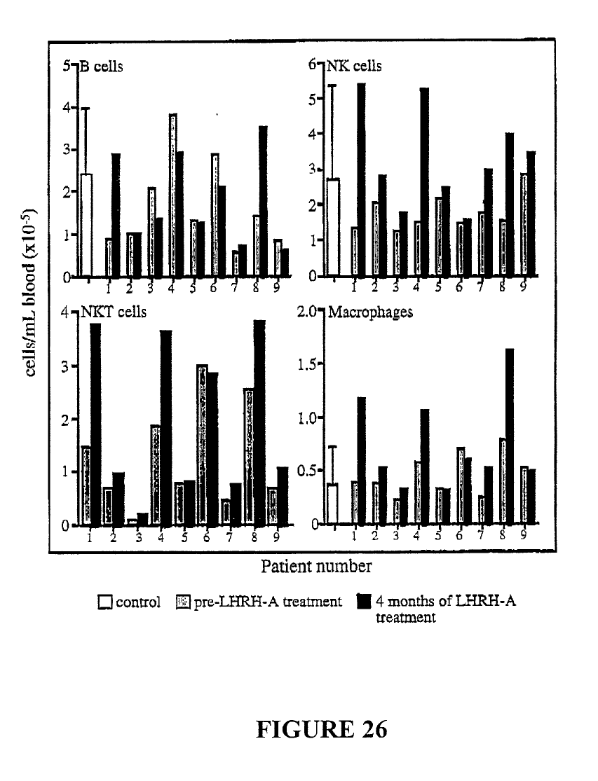

Analysis of the total cell numbers of B and myeloid cells within the peripheral blood post-treatment showed clearly increased levels of NK (5/9 patients), NKT (4/9 patients) and macrophage (3/9 patients) cell numbers post-treatment (FIG. 26). B cell numbers showed no distinct trend with 2/9 patients showing increased levels; 4/9 patients showing no change and 3/9 patients showing decreased levels.

The Effect Of LHRH Therapy On The Level Of Naive Cells Relative To Memory Cells:

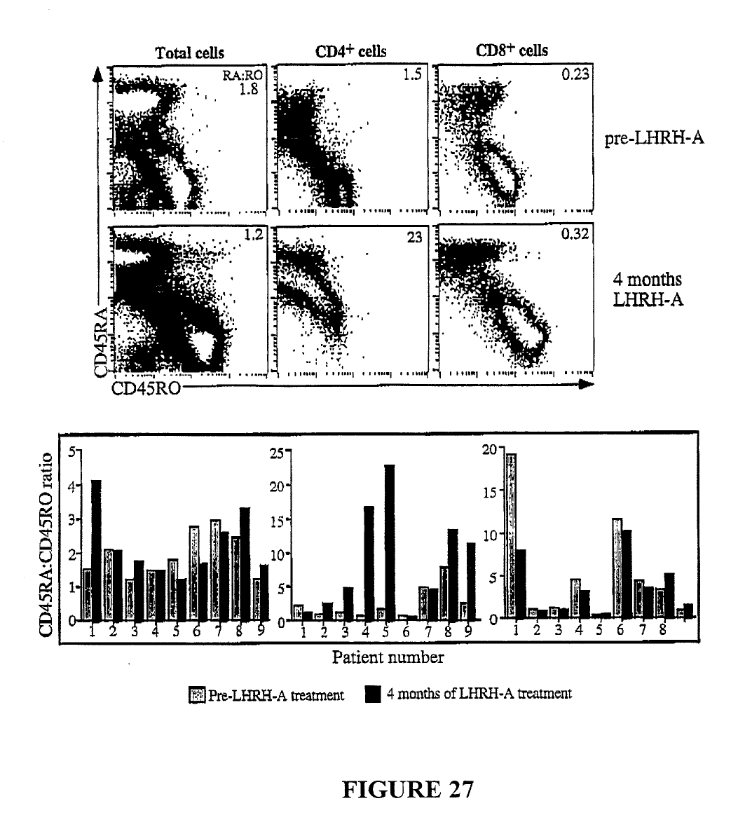

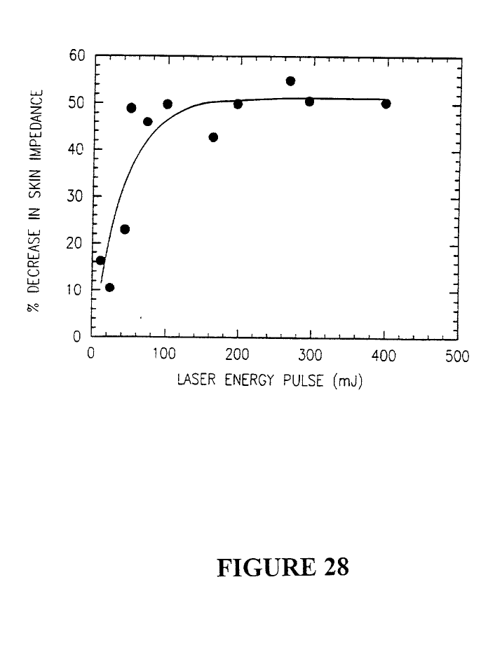

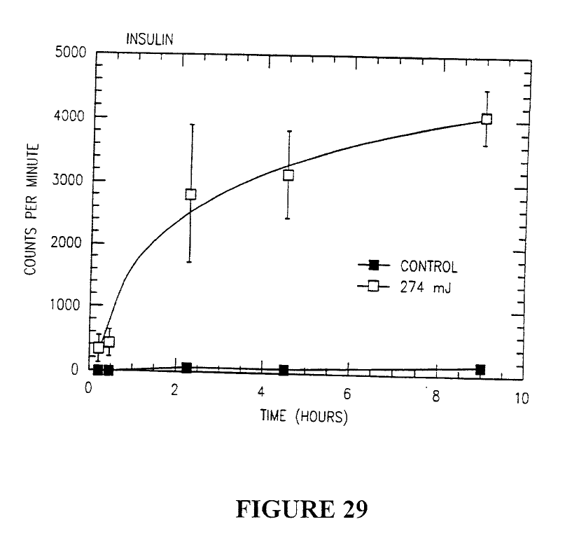

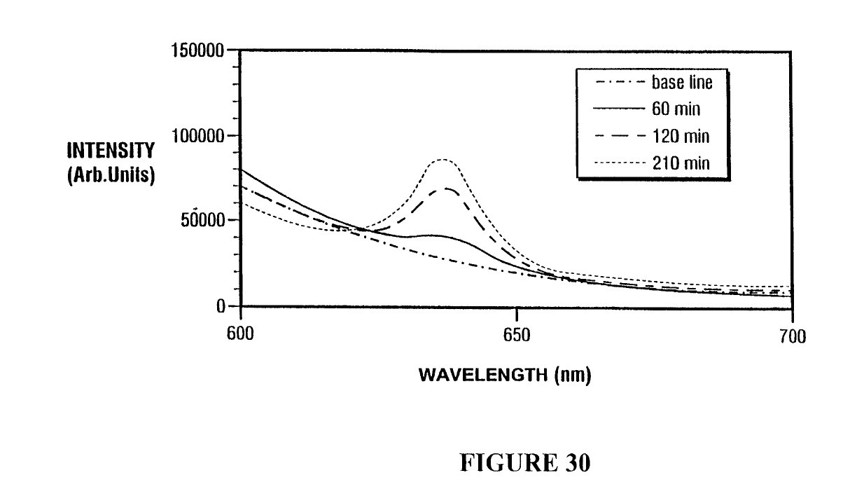

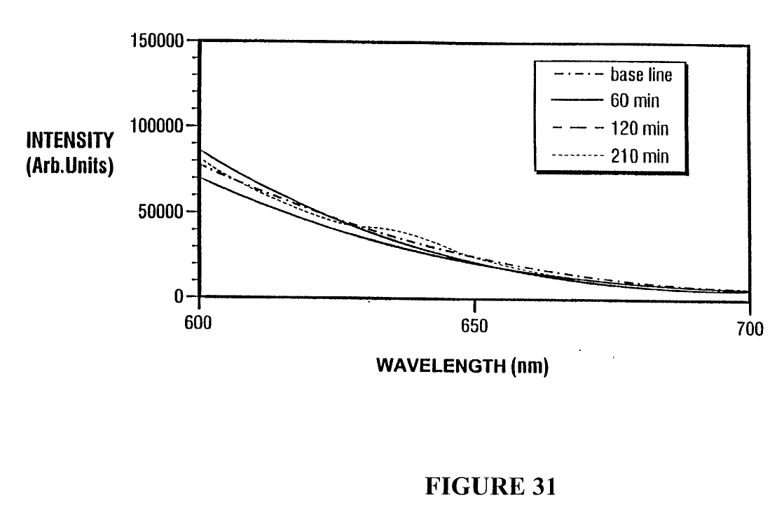

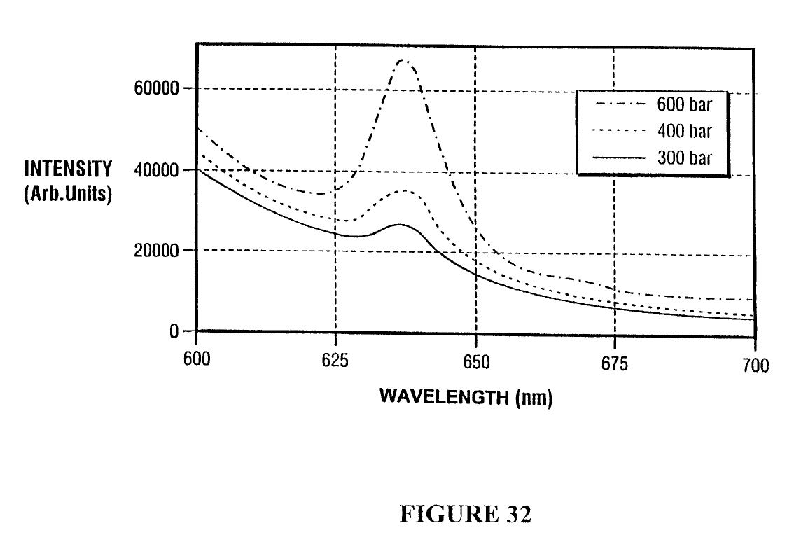

The major changes seen post-LHRH agonist treatment were within the T cell population of the peripheral blood. In particular there was a selective increase in the proportion of naive (CD45RA^{+) CD}4⁺ cells, with the ratio of naïve (CD45RA⁺) to memory (CD45RO⁺) in the CD4⁺ T cell subset increasing in 6/9 patients (FIG. 27).

Thus it can be concluded that LHRH agonist treatment of a human having an atrophied thymus can induce regeneration of the thymus. A general improvement has been shown in the status of blood T lymphocytes in these prostate cancer patients who have received sex-steroid ablation therapy. While it is very difficult to precisely determine whether such cells are only derived from the thymus, this would be very much the logical conclusion as no other source of mainstream (CD8 αβ chain) T cells has been described. Gastrointestinal tract T cells are predominantly TCR γδ or CD8 αα chain.

1. A method for improving vaccination comprising reactivation of the thymus of a patient.

2. The method of claim 1 wherein the patient's thymus has been at least in part deactivated.

3. The method of claim 2 wherein the patient is post-pubertal.

4. The method of claim 1 further comprising the step of administering hematopoietic stem cells to the patient.

5. The method of claim 4 wherein the hematopoietic stem cells are CD34+.

6. The method of claim 4 wherein the hematopoietic stem cells are autologous or syngeneic.

7. The method of claim 4 wherein the hematopoietic stem cells are allogeneic or xenogeneic.

8. The method of claim 4 wherein the hematopoietic stem cells are administered about the time when the thymus begins to regenerate or shortly thereafter.

9. The method of claim 4 wherein the hematopoietic stem cells are provided at the time disruption of sex steroid signaling to the thymus is begun.

10. The method of claim I wherein the method of disrupting the sex steroid signaling to the thymus is through surgical castration to remove the patient's gonads.

11. The method of claim 1 wherein the method of disrupting the sex steroid signaling to the thymus is through administration of one or more pharmaceuticals.

12. The method of claim 11 wherein the pharmaceuticals are selected from the group consisting of LHRH agonists, LHRH antagonists, anti-LHRH vaccines and combinations thereof.

13. The method of claim 12 wherein the LHRH agonists are selected from the group consisting of Eulexin, Goserelin, Leuprolide, Dioxalan derivatives, Triptorelin, Meterelin, Buserelin, Histrelin, Nafarelin, Lutrelin, Leuprorelin and Deslorelin.

14. The method of claim 1 resulting in a vaccine response by the patient's immune system that is comparable to the response of a pre-pubertal patient.
